(19) United States
(12) Patent Application Publication
(10) Pub. No.: US 2023/0417774 A1
(43) Pub. Date: Dec. 28, 2023

(54) METHOD FOR DETECTING MILD COGNITIVE IMPAIRMENT AND MILD ALZHEIMER'S DISEASE

(71) Applicant: The University of Tokyo, Tokyo (JP)

(72) Inventors: Yoshiya ODA, Tokyo (JP); Suzumi TOKUOKA, Tokyo (JP); Fumie HAMANO, Tokyo (JP)

(21) Appl. No.: 18/254,579

(22) PCT Filed: Nov. 26, 2021
(86) PCT No.: PCT/JP2021/043496

§ 371 Date: May 25, 2023

(30) Foreign Application Priority Data
Nov. 27, 2020 (JP) 2020-196825

(51) Int. Cl. G01N 33/68

(52) U.S. Cl. CPC G01N 33/6896; G01N 2800/56; G01N 2800/2821

(57) ABSTRACT

A method for detecting mild cognitive impairment and/or mild Alzheimer's disease, includes measuring the amount or concentration of a biomolecule in a biological sample from a test subject. The biomolecule is one or more proteins selected from FGF-19, PLA2G10 and CPA2. In the detection method, it is possible to determine the likelihood of suffering from mild cognitive impairment and/or mild Alzheimer's disease using the amount or concentration of the biomolecule in the biological sample from the test subject as an index.

### CROSS-REFERENCE TO RELATED APPLICATION

The present application enjoys the benefit of priority from the prior Japanese Patent Application No. 2020-196825 filed on Nov. 27, 2020, the entire disclosure of which is incorporated herein by reference.

### BACKGROUND OF THE INVENTION

### Technical Field

The present invention relates to a method for detecting mild cognitive impairment and mild Alzheimer's disease. The present invention also relates to a method for determining a therapeutic effect on mild cognitive impairment and mild Alzheimer's disease.

### Background Art

Currently, it is estimated that there are around 6 million patients with dementia in Japan, and that there are also around 4 million patients with mild cognitive impairment (MCI), which is a preliminary stage thereto. Dementia is often Alzheimer's dementia. When people are diagnosed as mild cognitive impairment, there are known cases of maintaining mild cognitive impairment, cases of returning to normal, cases of transitioning to Alzheimer's disease, cases of transitioning to frontotemporal dementia, and cases of transitioning to dementia with Lewy bodies. Once people develop Alzheimer's disease, they never return to normal, and absolute treatment to stop the progress of its symptoms has not yet been found. Therefore, it is desirable to find out a decline in cognitive function as early as possible.

So far, a method for examining dementia and/or mild cognitive impairment using a biomarker in a urine specimen has been proposed (Patent Document 1). However, the mainstream diagnosis of MCI and mild Alzheimer's disease is diagnosis based on subjective indexes such as medical interviews, diagnostic imaging which can be made only at limited facilities, and diagnosis based on highly invasive cerebrospinal fluid sampling. Therefore, it is desirable to be able to easily examine a decline in cognitive function.

### REFERENCE LIST

### Patent Document

Patent Document 1: JP 2016-148619 A

### SUMMARY OF THE INVENTION

An object of the present invention is to provide a novel method for detecting mild cognitive impairment and mild Alzheimer's disease. Another object of the present invention is to provide a novel method for determining a therapeutic effect on mild cognitive impairment and mild Alzheimer's disease.

The present inventors classified blood specimens from the National Center for Geriatrics and Gerontology (NCGG) biobank into a normal cognitive function (NC) group, a stable mild cognitive impairment (sMCI) group, and a progressive mild cognitive impairment (pMCI) group, and a mild Alzheimer's disease (AD) group, and performed proteomics. The present inventors also classified the blood specimens into the normal cognitive function (NC) group, a mild cognitive impairment (MCI) group, and the mild Alzheimer's disease (AD) group, and performed proteomics. As a result, when the measurement results of the sMCI group, pMCI group, MCI group and AD group were compared with those of the NC group, significant differences were confirmed in seven proteins (FGF-19, PLA2G10, CPA2, TIMP4, IGFBP1, NEFL and TMPRSS15). Also, the present inventors have found that mild cognitive impairment and mild Alzheimer's disease can be detected by using the amounts of the three proteins (FGF-19, PLA2G10 and CPA2) in the blood as indexes. Further, the present inventors have found that mild cognitive impairment and mild Alzheimer's disease can be detected more accurately by using combinations of these three proteins with known Alzheimer's disease markers (TIMP4, IGFBP1, NEFL and TMPRSS15) as indexes. The present inventors also performed lipid analysis using the above blood specimens. As a result, when the measurement results of the sMCI group, pMCI group, MCI group, and AD group were compared with those of the NC group, a significant difference was confirmed in a triglyceride free of fatty acid containing two or more unsaturated bonds. The present inventors have also found that mild cognitive impairment and mild Alzheimer's disease can be detected by using the amount of the triglyceride in the blood as an index; and further that mild cognitive impairment and mild Alzheimer's disease can be detected more accurately by using combinations of the above triglyceride with the seven protein markers described above as indexes. The present invention is based on these findings.

The present invention provides the following inventions.

[1] A method for detecting mild cognitive impairment and/or mild Alzheimer's disease, comprising the step of measuring the amount or concentration of a biomolecule in a biological sample from a test subject, wherein the biomolecule is one or more proteins (biomolecule (a1)) selected from the group consisting of FGF-19, PLA2G10 and CPA2.

[2] The detection method according to [1], further comprising the step of measuring the amount or concentration of a biomolecule other than the biomolecule (a1) in the biological sample from the test subject.

[3] The detection method according to [2], wherein the biomolecule other than the biomolecule (a1) is a protein and/or a lipid.

[4] The detection method according to [3], wherein the protein is one or more proteins (biomolecule (a2)) selected from the group consisting of TIMP4, IGFBP1, NEFL and TMPRSS15.

[5] The detection method according to [3], wherein the lipid is a triglyceride (biomolecule (b1)) free of fatty acid containing two or more unsaturated bonds, preferably one or more lipids selected from the group consisting of triglycerides listed in Table 1.

[6] A method for detecting mild cognitive impairment and/or mild Alzheimer's disease, comprising the step of measuring the amount or concentration of a biomolecule in a biological sample from a test subject, wherein the biomolecule is a triglyceride (biomolecule (b1)) free of fatty acid containing two or more unsaturated bonds, preferably one or more lipids selected from the group consisting of triglycerides listed in Table 1.

[7] The detection method according to [6], further comprising the step of measuring the amount or concentration of a biomolecule other than the biomolecule (b1) in the biological sample from the test subject.

[8] The detection method according to [7], wherein the biomolecule other than the biomolecule (b1) is a protein and/or a lipid.

[9] The detection method according to [8], wherein the protein is one or more proteins (biomolecule (a2)) selected from the group consisting of TIMP4, IGFBP1, NEFL and TMPRSS15.

[10] The detection method according to any one of [1] to [9], further comprising the step of determining the likelihood of suffering from mild cognitive impairment and/or mild Alzheimer's disease using the amount or concentration of the biomolecule in the biological sample from the test subject as an index.

[11] The detection method according to any one of [1] to [10], wherein the biological sample is a blood sample.

[12] A method for determining a therapeutic effect on mild cognitive impairment and/or mild Alzheimer's disease, comprising the step of measuring the amount or concentration of a biomolecule in a biological sample from a test subject, wherein the biomolecule is one or more proteins (biomolecule (a1)) selected from the group consisting of FGF-19, PLA2G10 and CPA2.

[13] The determination method according to [12], further comprising the step of measuring the amount or concentration of a biomolecule other than the biomolecule (a1) in the biological sample from the test subject.

[14] The determination method according to [13], wherein the biomolecule other than the biomolecule (a1) is a protein and/or a lipid.

[15] The determination method according to [14], wherein the protein is one or more proteins (biomolecule (a2)) selected from the group consisting of TIMP4, IGFBP1, NEFL and TMPRSS15.

[16] The determination method according to [14], wherein the lipid is a triglyceride (biomolecule (b1)) free of fatty acid containing two or more unsaturated bonds, preferably one or more lipids selected from the group consisting of triglycerides listed in Table 1.

[17] A method for determining a therapeutic effect on mild cognitive impairment and/or mild Alzheimer's disease, comprising the step of measuring the amount or concentration of a biomolecule in a biological sample from a test subject, wherein the biomolecule is a triglyceride (biomolecule (b1)) free of fatty acid containing two or more unsaturated bonds, preferably one or more lipids selected from the group consisting of triglycerides listed in Table 1.

[18] The determination method according to [17], further comprising the step of measuring the amount or concentration of a biomolecule other than the biomolecule (b1) in the biological sample from the test subject.

[19] The determination method according to [18], wherein the biomolecule other than the biomolecule (b1) is a protein and/or a lipid.

[20] The determination method according to [19], wherein the protein is one or more proteins (biomolecule (a2)) selected from the group consisting of TIMP4, IGFBP1, NEFL and TMPRSS15.

[21] The determination method according to any one of to [20], further comprising the step of determining the degree of the therapeutic effect on mild cognitive impairment and/or mild Alzheimer's disease using the amount or concentration of the biomolecule in the biological sample from the test subject as an index.

[22] The determination method according to any one of to [21], wherein treatment for mild cognitive impairment and/or mild Alzheimer's disease is drug therapy, diet therapy and/or exercise therapy.

[23] A method for determining the progress state of the pathological condition of mild cognitive impairment and/or mild Alzheimer's disease, comprising the step of measuring the amount or concentration of a biomolecule in a biological sample from a test subject, wherein the biomolecule is one or more proteins (biomolecule (a1)) selected from the group consisting of FGF-19, PLA2G10 and CPA2.

[24] The determination method according to [23], further comprising the step of measuring the amount or concentration of a biomolecule other than the biomolecule (a1) in the biological sample from the test subject.

[25] The determination method according to [24], wherein the biomolecule other than the biomolecule (a1) is a protein and/or a lipid, preferably wherein the protein is one or more proteins (biomolecule (a2)) selected from the group consisting of TIMP4, IGFBP1, NEFL and TMPRSS15, preferably wherein the lipid is a triglyceride (biomolecule (b1)) free of fatty acid containing two or more unsaturated bonds, and more preferably wherein the lipid is one or more lipids selected from the group consisting of triglycerides listed in Table 1.

[26] A method for determining the progress state of the pathological condition of mild cognitive impairment and/or mild Alzheimer's disease, comprising the step of measuring the amount or concentration of a biomolecule in a biological sample from a test subject, wherein the biomolecule is a triglyceride (biomolecule (b1)) free of fatty acid containing two or more unsaturated bonds, and preferably wherein the lipid is one or more lipids selected from the group consisting of triglycerides listed in Table 1.

[27] The determination method according to [26], further comprising the step of measuring the amount or concentration of a biomolecule other than the biomolecule (b1) in the biological sample from the test subject.

[28] The determination method according to [27], wherein the biomolecule other than the biomolecule (b1) is a protein and/or a lipid, and preferably wherein the protein is one or more proteins (biomolecule (a2)) selected from the group consisting of TIMP4, IGFBP1, NEFL and TMPRSS15.

[29] The determination method according to any one of to [28], further comprising the step of determining the degree of the progress state of the pathological condition of mild cognitive impairment and/or mild Alzheimer's disease using the amount or concentration of the biomolecule in the biological sample from the test subject as an index.

[30] The determination method according to any one of to [29], wherein the biological sample is a blood sample.

[31] Use of one or more proteins (biomolecule (a1)) selected from the group consisting of FGF-19, PLA2G10 and CPA2 and/or a triglyceride (biomolecule (b1)) free of fatty acid containing two or more unsaturated bonds, as a biomarker for detection or diagnosis of mild cognitive impairment and/or mild Alzheimer's disease.

[32] A method for screening for a candidate for a therapeutic agent or relieving agent for mild cognitive impairment and/or mild Alzheimer's disease, comprising the steps of: administering to a subject a candidate for a therapeutic agent or relieving agent for mild cognitive impairment and/or mild Alzheimer's disease; and measuring the amount or concentration of a biomolecule in a biological sample from the subject, wherein the biomolecule is one or more proteins (biomolecule (a1)) selected from the group consisting of FGF-19, PLA2G10 and CPA2.

[33] The screening method according to [32], further comprising the step of measuring the amount or concentration of a biomolecule other than the biomolecule (a1) in the biological sample from the test subject.

[34] The screening method according to [33], wherein the biomolecule other than the biomolecule (a1) is a protein and/or a lipid, preferably wherein the protein is one or more proteins (biomolecule (a2)) selected from the group consisting of TIMP4, IGFBP1, NEFL and TMPRSS15, preferably wherein the lipid is a triglyceride (biomolecule (b1)) free of fatty acid containing two or more unsaturated bonds, and more preferably wherein the lipid is one or more lipids selected from the group consisting of triglycerides listed in Table 1.

[35] A method for screening for a candidate for a therapeutic agent or relieving agent for mild cognitive impairment and/or mild Alzheimer's disease, comprising the steps of: administering to a subject a candidate for a therapeutic agent or relieving agent for mild cognitive impairment and/or mild Alzheimer's disease; and measuring the amount or concentration of a biomolecule in a biological sample from the subject, wherein the biomolecule is a triglyceride (biomolecule (b1)) free of fatty acid containing two or more unsaturated bonds, and preferably wherein the lipid is one or more lipids selected from the group consisting of triglycerides listed in Table 1.

[36] The screening method according to [35], further comprising the step of measuring the amount or concentration of a biomolecule other than the biomolecule (b1) in the biological sample from the test subject.

[37] The screening method according to [36], wherein the biomolecule other than the biomolecule (b1) is a protein and/or a lipid, and preferably wherein the protein is one or more proteins (biomolecule (a2)) selected from the group consisting of TIMP4, IGFBP1, NEFL and TMPRSS15.

[38] The screening method according to any one of to [37], further comprising the step of determining the degree of a therapeutic effect of the candidate for a therapeutic agent or relieving agent on mild cognitive impairment and/or mild Alzheimer's disease using the amount or concentration of the biomolecule in the biological sample from the subject as an index.

[39] The screening method according to any one of to [38], wherein the biological sample is a blood sample.

[40] A kit for detection or diagnosis of mild cognitive impairment and/or mild Alzheimer's disease, comprising a means of quantifying one or more proteins (biomolecule (a1)) selected from the group consisting of FGF-19, PLA2G10 and CPA2 and/or a triglyceride (biomolecule (b1)) free of fatty acid containing two or more unsaturated bonds, in a biological sample from a test subject.

[41] The kit according to [40], further comprising a means of quantifying a biomolecule other than the biomolecule (a1) and the biomolecule (b1).

[42] The kit according to [41], wherein the biomolecule other than the biomolecule (a1) and the biomolecule (b1) is a protein and/or a lipid, and preferably wherein the protein is one or more proteins (biomolecule (a2)) selected from the group consisting of TIMP4, IGFBP1, NEFL and TMPRSS15.

[43] A method for treating mild cognitive impairment and/or mild Alzheimer's disease, comprising the steps of: performing the detection method according to any one of [1] to [11] to determine the likelihood of suffering from mild cognitive impairment and/or mild Alzheimer's disease for a test subject from whom a biological sample has been collected; and performing treatment for mild cognitive impairment and/or mild Alzheimer's disease on a subject determined to suffer from or to be likely to suffer from mild cognitive impairment and/or mild Alzheimer's disease.

The present invention is advantageous in that mild cognitive impairment and mild Alzheimer's disease as well as therapeutic effects on mild cognitive impairment and mild Alzheimer's disease can be easily and accurately detected.

### BRIEF DESCRIPTION OF THE DRAWINGS

FIG. 1A is a diagram showing the results of measuring the relative quantification values of 368 proteins in the plasma for the sMCI group, pMCI group and AD group to the value for the NC group. Remarkably significant differences were confirmed in relative quantification values of six proteins (FGF-19, PLA2G10, CPA2, TIMP4, IGFBP1, and NEFL) (p<0.00001, One-Way ANOVA). FIG. 1B is a diagram showing the results of measuring the relative quantification values of 602 proteins in the plasma for the sMCI group, pMCI group and AD group to the value for the NC group. Remarkably significant differences were confirmed in relative quantification values of seven proteins (FGF-19, PLA2G10, CPA2, TIMP4, IGFBP1, NEFL, and TMPRSS15) (p<0.0000001, One-Way ANOVA).

FIG. 2 is a diagram showing the results of measuring the relative quantification values of 602 proteins in the plasma for the MCI group and AD group to the value for the NC group. Remarkably significant differences were confirmed in relative quantification values of seven proteins (FGF-19, PLA2G10, CPA2, TIMP4, IGFBP1, NEFL, and TMPRSS15) (p<0.00001, One-Way ANOVA).

FIG. 3 is a box plot created based on the relative quantification values of FGF-19 protein concentration in the plasma for the NC group, sMCI group, pMCI group, and AD group.

FIG. 4 is a box plot created based on the relative quantification values of CPA2 protein concentration in the plasma for the NC group, sMCI group, pMCI group, and AD group.

FIG. 5 is a box plot created based on the relative quantification values of PLA2G10 protein concentration in the plasma for the NC group, sMCI group, pMCI group, and AD group.

FIG. 6 is a box plot created based on the relative quantification values of TIMP4 protein concentration in the plasma for the NC group, sMCI group, pMCI group, and AD group.

FIG. 7 is a box plot created based on the relative quantification values of IGFBP1 protein concentration in the plasma for the NC group, sMCI group, pMCI group, and AD group.

FIG. 8 is a box plot created based on the relative quantification values of NEFL protein concentration in the plasma for the NC group, sMCI group, pMCI group, and AD group.

FIG. 9 is a box plot created based on the relative quantification values of TMPRSS15 protein concentration in the plasma for the NC group, sMCI group, pMCI group, and AD group.

FIG. 10 is a box plot created based on the relative quantification values of FGF-19 protein concentration in the plasma for the NC group, MCI group, and AD group.

FIG. 11 is a box plot created based on the relative quantification values of CPA2 protein concentration in the plasma for the NC group, MCI group, and AD group.

FIG. 12 is a box plot created based on the relative quantification values of PLA2G10 protein concentration in the plasma for the NC group, MCI group, and AD group.

FIG. 13 is a box plot created based on the relative quantification values of TIMP4 protein concentration in the plasma for the NC group, MCI group, and AD group.

FIG. 14 is a box plot created based on the relative quantification values of IGFBP1 protein concentration in the plasma for the NC group, MCI group, and AD group.

FIG. 15 is a box plot created based on the relative quantification values of NEFL protein concentration in the plasma for the NC group, MCI group, and AD group.

FIG. 16 is a box plot created based on the relative quantification values of TMPRSS15 protein concentration in the plasma for the NC group, MCI group, and AD group.

FIG. 17 shows ROC curves concerning discrimination between NC and sMCI when biomarkers (proteins) are used singly (corresponding to Table 2 in Example 2).

FIG. 18 shows an ROC curve concerning discrimination between NC and MCI when a biomarker (protein) is used singly (corresponding to a part of Table 3 in Example 2).

FIG. 19 shows ROC curves concerning discrimination between NC and AD when biomarkers (proteins) are used singly (corresponding to Table 4 in Example 2).

FIG. 20-1 shows ROC curves concerning discrimination between NC and sMCI when biomarkers (proteins) are used in combination (corresponding to a part of Table 5 in Example 2).

FIG. 20-2 is a continuation of FIG. 20-1.

FIG. 20-3 is a continuation of FIG. 20-2.

FIG. 21 shows ROC curves concerning discrimination between NC and MCI when biomarkers (proteins) are used in combination (corresponding to a part of Table 6 in Example 2).

FIG. 22-1 shows ROC curves concerning discrimination between NC and AD when biomarkers are used in combination (corresponding to Table 7 in Example 2).

FIG. 22-2 is a continuation of FIG. 22-1.

FIG. 22-3 is a continuation of FIG. 22-2.

FIG. 23 is a diagram showing the results of measuring the relative quantification values of 725 lipids in the plasma for the sMCI group, pMCI group and AD group to the value for the NC group. Remarkably significant differences were confirmed in relative quantification values of 18 lipids (p<0.00005, One-Way ANOVA).

FIG. 24 is a diagram showing the results of measuring the relative quantification values of 725 lipids in the plasma for the MCI group and AD group to the value for the NC group. Remarkably significant differences were confirmed in relative quantification values of 18 lipids (p<0.00005, One-Way ANOVA).

FIG. 25-1 is a box plot created based on the relative quantification value of each lipid in the plasma for the NC group, sMCI group, pMCI group, and AD group.

FIG. 25-2 is a continuation of FIG. 25-1.

FIG. 25-3 is a box plot created based on the relative quantification value of each lipid in the plasma for the NC group, MCI group, and AD group.

FIG. 25-4 is a continuation of FIG. 25-3.

FIG. 26 shows an ROC curve concerning discrimination between NC and sMCI when a biomarker (lipid) is used singly (corresponding to a part of Table 9 in Example 4).

FIG. 27 shows an ROC curve concerning discrimination between NC and MCI when a biomarker (lipid) is used singly (corresponding to a part of Table 10 in Example 4).

FIG. 28 shows an ROC curve concerning discrimination between NC and sMCI when biomarkers (lipids) are used in combination (corresponding to a part of Table 11 in Example 4).

FIG. 29 shows an ROC curve concerning discrimination between NC and MCI when biomarkers (lipids) are used in combination (corresponding to a part of Table 12 in Example 4).

FIG. 30 shows an ROC curve concerning discrimination between NC and sMCI when biomarkers (protein and lipid) are used in combination (corresponding to a part of Table 13 in Example 5).

FIG. 31 shows an ROC curve concerning discrimination between NC and MCI when biomarkers (proteins and lipid) are used in combination (corresponding to a part of Table 14 in Example 5).

### DETAILED DESCRIPTION OF THE INVENTION

### Definition

In the present invention, “FGF-19” means fibroblast growth factor-19; “PLA2G10” means Group X secretory phospholipase A2; “CPA2” means carboxypeptidase A2; “TIMP4” means tissue inhibitor of metalloproteinase 4; “IGFBP1” means Insulin-like growth factor-binding protein 1; “NEFL” means Neurofilament light polypeptide; and “TMPRSS15” means Transmembrane Protease, Serine 15, respectively. The sequence information on and isolation/purification methods for these proteins are publicly known, and those skilled in the art may prepare these proteins according to conventional methods, or may use commercially available products.

In the present invention, “mild cognitive impairment” is a condition with impairment in memory and the main symptom of forgetfulness, without obvious impairment in cognitive function other than decline in memory, and with no or, even if present, slight impact on daily life, which condition cannot be diagnosed as dementia (amnestic MCI), or a condition with the main symptom of decline in thinking ability (planning, organizing, and judgment), with no obvious impairment in cognitive function other than decline in thinking ability, and with no or, even if present, slight impact on daily life, which condition cannot be diagnosed as dementia (non-amnestic MCI).

In the present invention, “stable mild cognitive impairment” (stable MCI) refers to a case in which the condition of MCI has been maintained for 3 years or more after the diagnosis of MCI, and the transition to Alzheimer's disease has not been able to be confirmed. In the present invention, “progressive mild cognitive impairment” (progressive MCI) refers to a case in which the transition to Alzheimer's disease has been confirmed within 5 years after the diagnosis of MCI.

In the present invention, “mild Alzheimer's disease” is a condition in which complaints of memory impairment are expressed from persons themselves or their family members, impairment in one or more cognitive functions (memory, orientation, etc.) is objectively observed, and activities of daily living are normal, which condition cannot be diagnosed as dementia, and also refers to a case in which the result of a mini-mental state examination (MMSE examination, dementia screening examination) was 22 or 23.

In the present invention, the term “biological sample” means a sample separated from a living body, and is, for example, a body fluid such as blood, plasma, saliva, urine, cerebrospinal fluid, nasal discharge, sweat, tears, feces, or the like, preferably a blood sample (serum, plasma, etc.). The method for collecting the biological sample may be invasive or non-invasive, and can be selected according to the types of test subject and sample.

The “subject” in the present invention includes mammals including a human, and is preferably a human.

### <<Detection Method>>

According to a first aspect of the present invention, a method for detecting mild cognitive impairment and/or mild Alzheimer's disease is provided. According to the detection method of the present invention, it is possible to detect mild cognitive impairment and/or mild Alzheimer's disease using the amount or concentration of a biomolecule in a biological sample from a test subject as an index. That is, the detection method of the present invention is characterized by correlating the amount or concentration of the biomolecule with the likelihood of suffering from mild cognitive impairment and/or mild Alzheimer's disease in the test subject.

The biomolecule used as the index in the present invention is a specific protein or a specific lipid. That is, the detection method of the present invention can be divided into a mode using a specific protein as the index and a mode using a specific lipid as the index. The above specific protein may be combined with any other biomolecule than the protein. The above specific lipid may also be combined with any other biomolecule than the lipid.

When the biomolecule used as the index in the present invention is a specific protein, the protein contains at least one or more molecules selected from the group consisting of FGF-19, PLA2G10 and CPA2. In the present specification, the “one or more proteins selected from the group consisting of FGF-19, PLA2G10 and CPA2” is sometimes referred to as “biomolecule (a1) of the present invention” or “biomolecule (a1).”

When the biomolecule used as the index in the present invention is a specific protein, a biomolecule other than the biomolecule (a1) of the present invention (any other biomolecule) can be used, in addition to the biomolecule (a1) of the present invention, as the index of mild cognitive impairment and/or mild Alzheimer's disease. As the biomolecule other than the biomolecule (a1) of the present invention, any biomolecule may be used as long as it is used as the index of dementia (including mild cognitive impairment and mild Alzheimer's disease) (e.g., protein, peptide, lipid, nucleotide, and amino acid). Preferably, the biomolecule is, for example, either a protein or a lipid, or a combination thereof. The protein includes “TIMP4,” “IGFBP1,” “NEFL” and “TMPRSS15.” That is, in the present invention, one or more proteins (biomolecule (a2)) selected from the group consisting of “TIMP4”, “IGFBP1”, “NEFL” and “TMPRSS15” can be used in combination with the biomolecule (a1) of the present invention. In addition, the lipid is, for example, a triglyceride, and is preferably a triglyceride (biological molecule (b1)) free of fatty acid containing two or more unsaturated bonds (double bonds) (sometimes referred to as “polyunsaturated fatty acid”). Examples of such a triglyceride include triglycerides listed in Table 1 below. That is, in the present invention, the triglyceride (preferably, biomolecule (b1)) can be used in combination with the biomolecule (a1) of the present invention. More preferably, one or more lipids selected from the group consisting of triglycerides listed in Table 1 can be used as the biomolecule (b1).

When the biomolecule used as the index in the present invention is a specific lipid, the lipid includes at least a triglyceride (biological molecule (b1)) free of fatty acid containing two or more unsaturated bonds (double bonds) (polyunsaturated fatty acid). In the present specification, the “triglyceride free of fatty acid containing two or more unsaturated bonds” is sometimes referred to as “biomolecule (b1) of the present invention” or “biomolecule (b1).” Here, all fatty acids constituting the triglyceride as the biomolecule (b1) may either be free of unsaturated bond or have one unsaturated bond. In addition, all fatty acids constituting the triglyceride as the biomolecule (b1) may have 12 to 24 carbon atoms, preferably 14 to 22 carbon atoms. Further, the total number of unsaturated bonds of three fatty acids constituting the triglyceride as the biomolecule (b1) can be 0 to 2. Also, the total number of carbon atoms of three fatty acids constituting the triglyceride as the biomolecule (b1) can be 44 to 60, preferably 46 to 58.

Of the biomolecules that serve as the index in the present invention, the specific lipid may preferably be one or more lipids selected from the group consisting of triglycerides listed in Table 1.

TABLE 1
Biomarker (lipid) list

| | Charac- | Charac- | |
|---|---|---|---|
| | teristic 1 | teristic 2 | |
| Sym- | of triglyc- | of triglyc- | |
| bol | eride | eride | Explanation of triglyceride |
| A | TAG(58:1) | TG 22:0_36:1 | Component in which one of three |
| | | | fatty acids is fatty acid 22:0 (e.g., |
| | | | arachidic acid) |
| B | TAG(58:1) | TG 18:0_40:1 | Component in which one of three |
| | | | fatty acids is fatty acid 18:0 (e.g., |
| | | | stearic acid) |
| C | TAG(56:1) | TG 18:0_38:1 | Component in which one of three |
| | | | fatty acids is fatty acid 18:0 (e.g., |
| | | | stearic acid) |
| D | TAG(52:0) | TG 16:0_36:0 | Component in which one of three |
| | | | fatty acids is fatty acid 16:0 (e.g., |
| | | | palmitic acid) |
| E | TAG(54:0) | TG 22:0_32:0 | Component in which one of three |
| | | | fatty acids is fatty acid 22:0 (e.g., |
| | | | arachidic acid) |
| F | TAG(58:2) | TG 18:1_40:1 | Component in which one of three |
| | | | fatty acids is fatty acid 18:1 (e.g., |
| | | | oleic acid) |
| G | TAG(48:0) | TG 16:0_32:0 | Component in which one of three |
| | | | fatty acids is fatty acid 16:0 (e.g., |
| | | | palmitic acid) |
| H | TAG(52:0) | TG 18:0_34:0 | Component in which one of three |
| | | | fatty acids is fatty acid 18:0 (e.g., |
| | | | stearic acid) |
| I | TAG(48:0) | TG 18:0_30:0 | Component in which one of three |
| | | | fatty acids is fatty acid 18:0 (e.g., |
| | | | stearic acid) |
| J | TAG(56:1) | TG 16:0_40:1 | Component in which one of three |
| | | | fatty acids is fatty acid 16:0 (e.g., |
| | | | palmitic acid) |
| K | TAG(58:1) | TG 18:1_40:0 | Component in which one of three |
| | | | fatty acids is fatty acid 18:1 (e.g., |
| | | | oleic acid) |
| L | TAG(54:1) | TG 18:1_36:0 | Component in which one of three |
| | | | fatty acids is fatty acid 18:1 (e.g., |
| | | | oleic acid) |
| M | TAG(54:1) | TG 18:0_36:1 | Component in which one of three |
| | | | fatty acids is fatty acid 18:0 (e.g., |
| | | | stearic acid) |
| N | TAG(50:0) | TG 18:0_32:0 | Component in which one of three |
| | | | fatty acids is fatty acid 18:0 (e.g., |
| | | | stearic acid) |
| O | TAG(46:1) | TG 16:0_30:1 | Component in which one of three |
| | | | fatty acids is fatty acid 16:0 (e.g., |
| | | | palmitic acid) |
| P | TAG(48:0) | TG 14:0_34:0 | Component in which one of three |
| | | | fatty acids is fatty acid 14:0 (e.g., |
| | | | myristic acid) |
| Q | TAG(54:1) | TG 16:0_38:1 | Component in which one of three |
| | | | fatty acids is fatty acid 16:0 (e.g., |
| | | | palmitic acid) |
| R | TAG(50:0) | TG 16:0_34:0 | Component in which one of three |
| | | | fatty acids is fatty acid 16:0 (e.g., |
| | | | palmitic acid) |

* TAG(X:Y): X denotes the total number of carbon atoms of three fatty acids constituting triglycerides. Y denotes the number of double bonds contained in three fatty acids constituting triglycerides.
* TG A:B_C:D: “A:B” denotes the structure of one fatty acid among three fatty acids constituting triglycerides, in which A denotes the number of carbon atoms, and B denotes the number of double bonds. “C:D” denotes the structure of the remaining two fatty acids, in which C denotes the total number of carbon atoms of the two fatty acids, and D denotes the total number of double bonds of the two fatty acids.

Eighteen (18) lipids A to R listed in Table 1 above correspond to groups of lipids having structures characterized by the indications in Table 1, respectively. For example, lipid A will be explained as an example as follows. Specifically, one of the three fatty acids that bind to the triglyceride is a fatty acid (TG 22:0) with 22 carbon atoms and no unsaturated bond. This fatty acid includes branched-chain fatty acids in addition to arachidic acid which is a straight-chain fatty acid. In addition, the remaining two of the three fatty acids that bind to the triglyceride are fatty acids (TG 36:1) with 36 carbon atoms in total and one unsaturated bond in total, but can include any fatty acids as long as the two fatty acids satisfy the values (provided that the number of carbon atoms in one fatty acid can range from 12 to 24, as described above). In addition, the binding position of the fatty acid (TG 22:0), among the three fatty acids that bind to the triglyceride, in the triglyceride is unspecified, and can include any binding positions.

When the biomolecule used as the index in the present invention is a specific lipid, a biomolecule other than the biomolecule (b1) of the present invention (any other biomolecule) can be used, in addition to the biomolecule (b1) of the present invention, as the index of mild cognitive impairment and/or mild Alzheimer's disease. As the biomolecule other than the biomolecule (b1) of the present invention, any biomolecule may be used as long as it is used as the index of dementia (including mild cognitive impairment and mild Alzheimer's disease) (e.g., protein, peptide, lipid, nucleotide, and amino acid). Preferably, the biomolecule is, for example, either a protein or a lipid, or a combination thereof. The protein includes “TIMP4,” “IGFBP1,” “NEFL” and “TMPRSS15.” That is, in the present invention, one or more proteins (biomolecule (a2)) selected from the group consisting of “TIMP4”, “IGFBP1”, “NEFL” and “TMPRSS15” can be used in combination with the biomolecule (b1) of the present invention.

In the detection method of the present invention, first, the step (A) of measuring the amount or concentration of the biomolecule (a1) of the present invention or the biomolecule (b1) of the present invention (hereinafter sometimes referred to as “the biomolecule of the present invention”) in the biological sample from the test subject is carried out. When any other biomolecule is used as the index in addition to the biomolecule of the present invention, the step (X) of measuring the amount or concentration of the other biomolecule in the biological sample from the test subject is carried out. The amounts and concentrations of the biomolecules can be measured by known methods, and, for example, measurement methods using substances that specifically bind to the biomolecules can be used. Substances that specifically bind to the biomolecules typically include antibodies, aptamers (e.g., nucleic acid aptamers and peptide aptamers), drugs, fatty acid-binding proteins, and fat accumulation-induced transmembrane proteins. When antibodies or aptamers are used as the substances that specifically bind to the biomolecules, the amounts or concentrations of the biomolecules can be measured by immunoassay, DNA array, quantitative PCR, and next-generation sequencer (NGS). Immunoassays are analytical methods that use a detectably labeled anti-biomolecule antibody, or a detectably labeled antibody (secondary antibody) against an anti-biomolecule antibody. Depending on the antibody labeling method, the immunoassays are classified into enzyme immunoassay (EIA or ELISA), radioimmunoassay (RIA), fluorescence immunoassay (FIA), fluorescence polarization immunoassay (FPIA), chemiluminescence immunoassay (CLIA), and the like, and the biomolecules can be detected or quantified by an absorption method, a fluorometric method, a fluorescence polarization method, a chemiluminescence method, a bioluminescence method, an electrical conductivity detection method, an electrochemical detection method, an enzymatic method, or a method utilizing radioactive substances, or a combination of these methods. The immunoassay can be performed using two oligonucleotide-attached antibodies recognizing different epitopes for one target protein, which are used in the Examples (Example 1) below. In this case, double-stranded DNA can be quantified by quantitative PCR, DNA array and next-generation sequencer (NGS).

The method for measuring the biomolecules in the detection method of the present invention is not limited to the immunoassay, and the biomolecules may be measured by a mass spectrometry method. Examples of the mass spectrometry method include liquid chromatography-mass spectrometry (LC-MS), liquid chromatography-tandem mass spectrometry (LC-MSMS), high-performance liquid chromatography-mass spectrometry (HPLC-MS), high-performance liquid chromatography-tandem mass spectrometry (HPLC-MSMS), capillary electrophoresis-mass spectrometry (CE-MS), liquid capillary electrophoresis-tandem mass spectrometry (CE-MSMS), gas chromatography-mass spectrometry (GC-MS), and gas chromatography-tandem mass spectrometry (GC-MSMS). When using high-performance liquid chromatography, it is preferable to use a column that enables simultaneous analysis of a plurality of biomolecules (for example, a reversed-phase column or an ion-exchange column).

The detection method of the present invention can further include the step (B) of determining the likelihood of suffering from mild cognitive impairment and/or mild Alzheimer's disease for the test subject from whom the biological sample has been collected, using the amount or concentration of the biomolecule of the present invention measured in step (A) as an index.

When the biomolecule of the present invention is the biomolecule (a1), the step (B) can be carried out by the step (B-a1-1) of comparing the amount or concentration of the biomolecule (a1) of the present invention in the biological sample from the test subject with a predetermined cutoff value; and the step (B-a1-2) of determining that the test subject suffers from (or is likely to suffer from) mild cognitive impairment and/or mild Alzheimer's disease, when the amount or concentration of the biomolecule (a1) of the present invention in the biological sample from the test subject is equal to or higher than the cutoff value or higher than the cutoff value. In the present invention, the meaning of the term “likelihood of suffering” includes “risk of developing dementia (including mild cognitive impairment and mild Alzheimer's disease)”.

Also, in the step (B-a1-2), when the amount or concentration of the biomolecule (a1) of the present invention in the biological sample from the test subject is equal to or lower than the cutoff value or lower than the cutoff value, it can be determined that the test subject does not suffer from (or is less likely to suffer from) mild cognitive impairment and/or mild Alzheimer's disease.

When the biomolecule of the present invention is the biomolecule (b1), the step (B) can be carried out by the step (B-b1-1) of comparing the amount or concentration of the biomolecule (b1) of the present invention in the biological sample from the test subject with a predetermined cutoff value; and the step (B-b1-2) of determining that the test subject suffers from (or is likely to suffer from) mild cognitive impairment and/or mild Alzheimer's disease, when the amount or concentration of the biomolecule (b1) of the present invention in the biological sample from the test subject is equal to or higher than the cutoff value or higher than the cutoff value.

Also, in the step (B-b1-2), when the amount or concentration of the biomolecule (b1) of the present invention in the biological sample from the test subject is equal to or lower than the cutoff value or lower than the cutoff value, it can be determined that the test subject does not suffer from (or is less likely to suffer from) mild cognitive impairment and/or mild Alzheimer's disease.

When two or more of the biomolecules of the present invention are used in combination for detection in the detection method of the present invention, the likelihood of suffering from mild cognitive impairment and/or mild Alzheimer's disease can be detected more accurately than when the biomolecule of the present invention is used singly for detection. When two or more of the biomolecules of the present invention are used in combination for detection in the detection method of the present invention, the step (A) and the step (B) can be performed on the respective biomolecules. In this case, the likelihood of suffering from the disease(s) can be determined by combining the likelihood of suffering from the disease(s) indicated based on the individual biomolecules. For example, when the likelihood of suffering from the disease(s) is indicated for both of the two biomolecules of the present invention, the likelihood of suffering from the disease(s) is suggested more strongly than in the results obtained using each biomolecule singly. When the likelihood of suffering from the disease(s) is denied (or when the likelihood of suffering from the disease(s) is indicated to be low) for both of the two biomolecules of the present invention, the likelihood of suffering from the disease(s) is denied more strongly than in the results obtained using each biomolecule singly.

Also, the detection method of the present invention can further include the step (P) of determining the likelihood of suffering from mild cognitive impairment and/or mild Alzheimer's disease for the test subject from whom the biological sample has been collected, using the amount or concentration of the other biomolecule measured in step (X) as an index. When the biomolecule of the present invention is the biomolecule (a1), the biomolecule (a2) and/or the biomolecule (b1) can be used as the other biomolecule(s). When the biomolecule of the present invention is the biomolecule (b1), the biomolecule (a2) can be used as the other biomolecule.

When the other biomolecule is the biomolecule (a2), the step (P) can be carried out by the step (P-a2-1) of comparing the amount or concentration of the biomolecule (a2) in the biological sample from the test subject with a predetermined cutoff value; and the step (P-a2-2) of determining that the test subject suffers from (or is likely to suffer from) mild cognitive impairment and/or mild Alzheimer's disease when the amount or concentration of any of TIMP4, NEFL and TMPRSS15 among the biomolecules (a2) in the biological sample from the test subject is equal to or higher than the cutoff value or higher than the cutoff value, and determining that the test subject suffers from (or is likely to suffer from) mild cognitive impairment and/or mild Alzheimer's disease when the amount or concentration of IGFBP1 among the biomolecules (a2) in the biological sample from the test subject is equal to or lower than the cutoff value or lower than the cutoff value.

In the step (P-a2-2), it is determined that the test subject does not suffer from (or is less likely to suffer from) mild cognitive impairment and/or mild Alzheimer's disease when the amount or concentration of any of TIMP4, NEFL and TMPRSS15 among the biomolecules (a2) in the biological sample from the test subject is equal to or lower than the cutoff value or lower than the cutoff value, and that the test subject does not suffer from (or is less likely to suffer from) mild cognitive impairment and/or mild Alzheimer's disease when the amount or concentration of IGFBP1 among the biomolecules (a2) in the biological sample from the test subject is equal to or higher than the cutoff value or higher than the cutoff value.

When the other biomolecule is the biomolecule (b1), the step (P) can be carried out by the step (P-b1-1) of comparing the amount or concentration of the biomolecule (b1) in the biological sample from the test subject with a predetermined cutoff value; and the step (P-b1-2) of determining that the test subject suffers from (or is likely to suffer from) mild cognitive impairment and/or mild Alzheimer's disease, when the amount or concentration of the biomolecule (b1) in the biological sample from the test subject is equal to or higher than the cutoff value or higher than the cutoff value.

Also, in the step (P-b1-2), when the amount or concentration of the biomolecule (b1) in the biological sample from the test subject is equal to or lower than the cutoff value or lower than the cutoff value, it can be determined that the test subject does not suffer from (or is less likely to suffer from) mild cognitive impairment and/or mild Alzheimer's disease.

When detection is performed using the other biomolecule in combination with the biomolecule of the present invention in the detection method of the present invention, the likelihood of suffering from mild cognitive impairment and/or mild Alzheimer's disease can be determined more accurately than when detection is performed using only the biomolecule of the present invention, as will be indicated in the Examples below. Therefore, the likelihood indicated in the step (P) can be used to supplement the likelihood indicated in the step (B). For example, when the likelihood of suffering from the disease(s) is indicated in the steps (B) and (P), the likelihood is suggested more strongly than in the results obtained in the step (B) alone. When the likelihood of suffering from the disease(s) is denied (or when the likelihood is indicated to be low) in the steps (B) and (P), the likelihood is denied more strongly than in the results obtained in the step (B) alone.

In the present invention, the cutoff value can be calculated and determined from the measured value of the amount or concentration of the biomolecule of the present invention in a sample from a subject (normal subject) who does not suffer from dementia including mild cognitive impairment and mild Alzheimer's disease. Such a subject is preferably a healthy individual having no disease being treated, but may be a subject having a disease other than dementia (including mild cognitive impairment and mild Alzheimer's disease). In the present invention, the cutoff value can also be calculated and determined from the measured value of the amount or concentration of the biomolecule of the present invention in a sample from a subject (affected subject) who suffers from mild cognitive impairment and/or mild Alzheimer's disease. An average value, median value, percentile value, maximum value or minimum value of the measured value for a normal subject group or affected subject group can be used in the method for measuring the cutoff value described above. The percentile value can be selected arbitrarily, and can be, for example, 5, 10, 15, 20, 25, 30, 40, 50, 60, 70, 75, 80, 85, 90 or 95. The numbers of normal subject and affected subject cases at the time of calculating the cutoff value are preferably be multiple, and can be, for example, 2 or more, 5 or more, 10 or more, 20 or more, 50 or more, or 100 or more.

In the present invention, the cutoff value can also be calculated and determined based on the measured value of the amount or concentration of the biomolecule of the present invention in the samples from the subjects (normal subjects) who do not suffer from dementia including mild cognitive impairment and mild Alzheimer's disease, and the measured value of the amount or concentration of the biomolecule of the present invention in the samples from the subjects (affected subjects) who suffer from mild cognitive impairment and/or mild Alzheimer's disease. For example, the cutoff value can be set by measuring the amount or concentration of the biomolecule of the present invention in a biological sample for the affected subject group and the normal subject group, and performing statistical analysis such as ROC (Receiver Operating Characteristic curve) analysis using the obtained measured values. The creation of the ROC curve and the setting of the cutoff value based on the ROC curve are well known, and can be appropriately set by those skilled in the art from the viewpoint of sensitivity and specificity.

In the detection method of the present invention, when the other biomolecule is used as the index in addition to the biomolecule of the present invention, the cutoff value of the other biomolecule can be calculated and determined according to the descriptions about the cutoff value of the biomolecule of the present invention.

In the step (B) of the detection method of the present invention, for example, when the concentration of the biomolecule of the present invention in the biological sample from the test subject is higher than the average value of the amount or concentration of the biomolecule in the normal subject group, or is about 1.05 times or more, about 1.1 times or more, about 1.2 times or more, about 1.3 times or more, about 1.4 times or more, about 1.5 times or more, about 1.6 times or more, about 1.7 times or more, about 1.8 times or more, about 1.9 times or more, about 2.0 times or more, about 2.5 times or more, or about 3 times or more the average value, it can be determined that the test subject suffers from (or is likely to suffer from) mild cognitive impairment and/or mild Alzheimer's disease.

In the present invention, detection accuracy can be improved by using a combination of a plurality of the biomolecules of the present invention. Also, in the present invention, detection accuracy can further be improved by using a combination of the biomolecule of the present invention with the other biomolecule. The phrase “detection accuracy is improved” as used herein means that the area under the curve (AUC) of the ROC curve is improved, when ROC analysis is utilized.

In the present invention, when a plurality of the biomolecules of the present invention are used in combination as the index, or when the biomolecule of the present invention is used in combination with the other biomolecule as the index, one cutoff value can also be set for the measured values of the amounts or concentrations of the plurality of biomolecules serving as the indexes. For example, the cutoff value can be calculated using the total value, average value, ratio or the like of the measured values of the amounts or concentrations of the plurality of biomolecules, instead of the measured value of the amount or concentration of one biomolecule. Alternatively, it is possible to weight the measured values of the amounts or concentrations of the plurality of biomolecules, thereafter to calculate the total value, average value, ratio or the like thereof, and to calculate the cutoff value using the calculated values. When the cutoff value calculated in this way is used in the present invention, determination can be performed by processing the measured values of the amounts or concentrations of the plurality of biomolecules in the biological sample from the test subject in the same manner as in the cutoff value calculation method, and comparing the obtained numerical values with the predetermined cutoff value.

According to the detection method of the present invention, it is possible to detect mild cognitive impairment and/or mild Alzheimer's disease for the test subject. Therefore, the detection method of the present invention can be used supplementarily in the diagnosis of mild cognitive impairment and/or mild Alzheimer's disease, and a doctor can finally judge whether the test subject suffers from mild cognitive impairment and/or mild Alzheimer's disease in combination with any other finding in some cases. For example, in the present invention, for the test subject determined to suffer from (or to be likely to suffer from) mild cognitive impairment and/or mild Alzheimer's disease, a doctor can finally judge whether the test subject suffers from (or is likely to suffer from) mild cognitive impairment or mild Alzheimer's disease, while referring to any other finding.

According to the detection method of the present invention, it is possible to quantitatively detect mild cognitive impairment and/or mild Alzheimer's disease based on the biological sample collected from the test subject. That is, the detection method of the present invention is advantageous in that the method enables easy and accurate detection of mild cognitive impairment and/or mild Alzheimer's disease while reducing the burden on patients. The detection method of the present invention enables detection based on a biological sample collected by a less invasive method such as blood collection, and is thus advantageous also in that the method enables detection of mild cognitive impairment and/or mild Alzheimer's disease at many facilities using biological samples obtained in a less invasive manner, as compared with conventional diagnosis methods such as highly invasive diagnosis methods such as cerebrospinal fluid sampling and diagnostic imaging methods that can be carried out only at limited facilities.

According to another aspect of the present invention, a method for diagnosing mild cognitive impairment and/or mild Alzheimer's disease is provided. According to the diagnosis method of the present invention, it is possible to carry out diagnosis as to whether a test subject suffers from mild cognitive impairment and/or mild Alzheimer's disease, using the amount or concentration of the biomolecule of the present invention in a biological sample from the test subject as an index. In the diagnosis method of the present invention, as in the detection method of the present invention, the step (A′) of measuring the concentration of the biomolecule of the present invention in the biological sample from the test subject is carried out. When any other biomolecule is used as the index in addition to the biomolecule of the present invention, the step (X′) of measuring the amount or concentration of the other biomolecule in the biological sample from the test subject is carried out. The diagnosis method of the present invention may further include the step (B′) of determining the likelihood of suffering from mild cognitive impairment and/or mild Alzheimer's disease for the test subject from whom the biological sample has been collected, using the amount or concentration of the biomolecule of the present invention measured in step (A′) as an index. When the biomolecule of the present invention is the biomolecule (a1), the step (B′) can be carried out by the step (B′-a1-1) of comparing the amount or concentration of the biomolecule (a1) of the present invention in the biological sample from the test subject with a predetermined cutoff value; and the step (B′-a1-2) of determining that the test subject suffers from (or is likely to suffer from) mild cognitive impairment and/or mild Alzheimer's disease, when the amount or concentration of the biomolecule (a1) of the present invention in the biological sample from the test subject is equal to or higher than the cutoff value or higher than the cutoff value. When the biomolecule of the present invention is the biomolecule (b1), the step (B′) can be carried out by the step (B′-b1-1) of comparing the amount or concentration of the biomolecule (b1) of the present invention in the biological sample from the test subject with a predetermined cutoff value; and the step (B′-b1-2) of determining that the test subject suffers from (or is likely to suffer from) mild cognitive impairment and/or mild Alzheimer's disease, when the amount or concentration of the biomolecule (b1) of the present invention in the biological sample from the test subject is equal to or higher than the cutoff value or higher than the cutoff value. The steps (A′), (B′), (B′-a1-1), (B′-a1-2), (B′-b1-1) and (B′-b1-2) correspond to the steps (A), (B), (B-a1-1), (B-a1-2), (B′-b1-1) and (B′-b1-2), respectively, and can be carried out according to the descriptions about the detection method of the present invention. In the diagnosis method of the present invention, as in the detection method of the present invention, any other biomolecule can be used as an index, in addition to the biomolecule of the present invention. In this case, the likelihood of suffering from mild cognitive impairment and/or mild Alzheimer's disease can be determined according to the descriptions about the steps (X) and (P) of the detection method of the present invention.

### <<Method for Determining Therapeutic Effect>>

According to a second aspect of the present invention, a method for detecting a therapeutic effect on mild cognitive impairment and/or mild Alzheimer's disease is provided. According to the method for determining a therapeutic effect of the present invention, it is possible to determine a therapeutic effect on mild cognitive impairment and/or mild Alzheimer's disease using the amount or concentration of the biomolecule of the present invention in a biological sample from a test subject as an index. That is, the method for determining a therapeutic effect of the present invention is characterized by correlating the amount or concentration of the biomolecule of the present invention with the therapeutic effect on mild cognitive impairment and/or mild Alzheimer's disease in the test subject.

In the method for determining a therapeutic effect of the present invention, as in the detection method of the present invention, the step (C) of measuring the amount or concentration of the biomolecule of the present invention in the biological sample from the test subject is carried out. When any other biomolecule is used as the index in addition to the biomolecule of the present invention, the step (Y) of measuring the amount or concentration of the other biomolecule in the biological sample from the test subject is carried out. The concentration of the biomolecule can be measured in the same manner as in the detection method of the present invention.

Since the method for determining a therapeutic effect of the present invention is intended to determine a therapeutic effect, the test subject can be a test subject after treatment or during treatment. Here, treatment for mild cognitive impairment and/or mild Alzheimer's disease includes lifestyle modifications such as drug therapy, diet therapy and exercise therapy. The drug therapy includes treatment via administration of a drug such as donepezil or memantine.

The method for determining a therapeutic effect of the present invention can further include the step (D) of determining the degree of the therapeutic effect on mild cognitive impairment and/or mild Alzheimer's disease for the subject who has undergone treatment, using the amount or concentration of the biomolecule of the present invention measured in step (C) as an index.

When the biomolecule of the present invention is the biomolecule (a1), the step (D) can be carried out by the step (D-a1-1) of comparing the amount or concentration of the biomolecule (a1) of the present invention in the biological sample from the test subject with a predetermined cutoff value; and the step (D-a1-2) of determining that the treatment is likely to be effective (or the treatment is highly likely to be effective), when the amount or concentration of the biomolecule (a1) of the present invention in the biological sample from the test subject is equal to or lower than the cutoff value or lower than the cutoff value.

Also, in the step (D-a1-2), when the amount or concentration of the biomolecule (a1) of the present invention in the biological sample from the test subject is equal to or higher than the cutoff value or higher than the cutoff value, it can be determined that the treatment is likely to be ineffective (or the treatment is less likely to be effective).

When the biomolecule of the present invention is the biomolecule (b1), the step (D) can be carried out by the step (D-b1-1) of comparing the amount or concentration of the biomolecule (b1) of the present invention in the biological sample from the test subject with a predetermined cutoff value; and the step (D-b1-2) of determining that the treatment is likely to be effective (or the treatment is highly likely to be effective), when the amount or concentration of the biomolecule (b1) of the present invention in the biological sample from the test subject is equal to or lower than the cutoff value or lower than the cutoff value.

Also, in the step (D-b1-2), when the amount or concentration of the biomolecule (b1) of the present invention in the biological sample from the test subject is equal to or higher than the cutoff value or higher than the cutoff value, it can be determined that the treatment is likely to be ineffective (or the treatment is less likely to be effective).

When two or more of the biomolecules of the present invention are used in combination for determination in the method for determining a therapeutic effect of the present invention, the degree of the therapeutic effect can be determined more accurately than when the biomolecule of the present invention is used singly for determination. When two or more of the biomolecules of the present invention are used in combination for determination in the method for determining a therapeutic effect of the present invention, the step (C) and the step (D) can be performed on the respective biomolecules. In this case, the degree of the therapeutic effect can be determined by combining the degrees of the therapeutic effect indicated based on the individual biomolecules. For example, when the likelihood that the treatment may be effective is indicated for both of the two biomolecules of the present invention, the likelihood that the treatment may be effective is suggested more strongly than in the results obtained using each biomolecule singly. When the likelihood that the treatment may be ineffective (or when the likelihood that the treatment may be effective is indicated to be low) for both of the two biomolecules of the present invention, the therapeutic effect is denied more strongly than in the results obtained using each biomolecule singly.

The method for determining a therapeutic effect of the present invention can further include the step (Q) of determining the degree of the therapeutic effect on mild cognitive impairment and/or mild Alzheimer's disease for the subject who has undergone treatment, using the amount or concentration of the other biomolecule measured in step (Y) as an index. When the biomolecule of the present invention is the biomolecule (a1), the biomolecule (a2) and/or the biomolecule (b1) can be used as the other biomolecule(s). When the biomolecule of the present invention is the biomolecule (b1), the biomolecule (a2) can be used as the other biomolecule.

When the other biomolecule is the biomolecule (a2), the step (Q) can be carried out by the step (Q-a2-1) of comparing the amount or concentration of the biomolecule (a2) in the biological sample from the test subject with a predetermined cutoff value; and the step (Q-a2-2) of determining that the treatment is likely to be effective (or the treatment is highly likely to be effective) when the amount or concentration of any of TIMP4, NEFL and TMPRSS15 among the biomolecules (a2) in the biological sample from the test subject is equal to or lower than the cutoff value or lower than the cutoff value, and determining that the treatment is likely to be effective (or the treatment is highly likely to be effective) when the amount or concentration of IGFBP1 among the biomolecules (a2) in the biological sample from the test subject is equal to or higher than the cutoff value or higher than the cutoff value.

In the step (Q-a2-2), it is determined that the treatment is likely to be ineffective (or the treatment is less likely to be effective) when the amount or concentration of any of TIMP4, NEFL and TMPRSS15 among the biomolecules (a2) in the biological sample from the test subject is equal to or higher than the cutoff value or higher than the cutoff value, and that the treatment is likely to be ineffective (or the treatment is less likely to be effective) when the amount or concentration of IGFBP1 among the biomolecules (a2) in the biological sample from the test subject is equal to or lower than the cutoff value or lower than the cutoff value.

When the other biomolecule is the biomolecule (b1), the step (Q) can be carried out by the step (Q-b1-1) of comparing the amount or concentration of the biomolecule (b1) in the biological sample from the test subject with a predetermined cutoff value; and the step (Q-b1-2) of determining that the treatment is likely to be effective (or the treatment is highly likely to be effective), when the amount or concentration of the biomolecule (b1) in the biological sample from the test subject is equal to or lower than the cutoff value or lower than the cutoff value.

Also, in the step (Q-b1-2), when the amount or concentration of the biomolecule (b1) in the biological sample from the test subject is equal to or higher than the cutoff value or higher than the cutoff value, it can be determined that the treatment is likely to be ineffective (or the treatment is less likely to be effective).

When detection is performed using the other biomolecule in combination with the biomolecule of the present invention in the method for determining a therapeutic effect of the present invention, the degree of the therapeutic effect can be determined more accurately than when detection is performed using only the biomolecule of the present invention, as will be indicated in the Examples below. Therefore, the therapeutic effect indicated in the step (Q) can be used to supplement the therapeutic effect indicated in the step (D). For example, when the likelihood that the treatment may be effective is indicated in the steps (D) and (Q), the likelihood that the treatment may be effective is suggested more strongly than in the results obtained in the step (D) alone. When the likelihood that the treatment may be ineffective (or when the likelihood that the treatment may be effective is indicated to be low) in the steps (D) and (Q), the therapeutic effect is denied more strongly than in the results obtained in the step (D) alone.

The cutoff value used in the method for determining a therapeutic effect of the present invention can be set according to the descriptions about the detection method of the present invention. In addition to this, measured values (reference values) of the amounts or concentrations of the biomolecule of the present invention and the other biomolecule in the biological sample from the test subject before the treatment can be used in place of the cutoff value in the method for determining a therapeutic effect of the present invention. In this case, the method for determining a therapeutic effect of the present invention may further include the step of measuring the amount or concentration of the biomolecule of the present invention and/or the other biomolecule in the biological sample from the test subject, before start of the treatment for mild cognitive impairment and/or mild Alzheimer's disease or after start thereof (preferably soon after start, e.g., within one to two days, within a few days, or within one week). In the case where the biomolecule is any of FGF-19, PLA2G10, CPA2, TIMP4, NEFL and TMPRSS15, it can be determined that the treatment is likely to be effective when the amount or concentration of the biomolecule in the biological sample from the test subject is lower than the reference value. In the case where the biomolecule is IGFBP1, it can be determined that the treatment is likely to be effective when the amount or concentration of the biomolecule in the biological sample from the test subject is higher than the reference value. Also, in the case where the biomolecule is biomolecule (b1), it can be determined that the treatment is likely to be effective when the amount or concentration of the biomolecule in the biological sample from the test subject is lower than the reference value.

In the method for determining a therapeutic effect of the present invention, the steps (C) and (D) or the steps (Y) and (Q) can be carried out after a certain period of time has passed after start of the treatment for mild cognitive impairment and/or mild Alzheimer's disease, or after a certain period of time has passed after the implementation of the treatment. The period of time from the start of the treatment or the implementation of the treatment to the implementation of the steps (C) and (D) or the steps (Y) and (Q) can be determined according to the expected therapeutic effect. For example, when the therapeutic effect is expected to be exerted about 1 to 2 months after start of the treatment, the steps (C) and (D) or the steps (Y) and (Q) can be carried out about one month or about two months after start of the treatment, and, subsequently, the steps (C) and (D) or the steps (Y) and (Q) may be carried out again after a period of time of about one month or about two months.

The subject who undergoes the treatment in the method for determining a therapeutic effect of the present invention is preferably a subject suffering from mild cognitive impairment and/or mild Alzheimer's disease. In the present invention, the “subject suffering from mild cognitive impairment and/or mild Alzheimer's disease” is, for example, a subject diagnosed by a doctor as developing mild cognitive impairment and/or mild Alzheimer's disease, and may be a subject whose results obtained by other examination methods suggest the possibility that the subject may suffer from mild cognitive impairment and/or mild Alzheimer's disease.

In the step (D) of the method for determining a therapeutic effect of the present invention, for example, when the amount or concentration of the biomolecule (a1) of the present invention in the biological sample from the subject who has undergone the treatment is about 1.3 times or less, about 1.2 times or less, about 1.1 times or less, or about 1.1 times or less the average value of the amount or concentration of the biomolecule for the normal subject group, it can be determined that the treatment is likely to be effective. Also, in the step (D) of the method for determining a therapeutic effect of the present invention, when the amount or concentration of the biomolecule (a1) of the present invention in the biological sample from the subject who has undergone the treatment is lower than the average value of the amount or concentration of the biomolecule for the affected subject group, or is about 0.95 times or less, about 0.9 times or less, about 0.8 times or less, or about 0.7 times or less the average value, it can be determined that the treatment is likely to be effective.

Also, in the step (D) of the method for determining a therapeutic effect of the present invention, when the amount or concentration of the biomolecule (11) of the present invention in the biological sample from the subject who has undergone the treatment is about 1.3 times or less, about 1.2 times or less, about 1.1 times or less, or about 1.05 times or less the average value of the amount or concentration of the biomolecule for the normal subject group, it can be determined that the treatment is likely to be effective. Also, in the step (D) of the method for determining a therapeutic effect of the present invention, when the amount or concentration of the biomolecule (b1) of the present invention in the biological sample from the subject who has undergone the treatment is lower than the average value of the amount or concentration of the biomolecule for the affected subject group, or is about 0.95 times or less, about 0.9 times or less, about 0.8 times or less, or about 0.7 times or less the average value, it can be determined that the treatment is likely to be effective.

According to the method for determining a therapeutic effect of the present invention, the therapeutic effect can be determined in the subject who has undergone the treatment for mild cognitive impairment and/or mild Alzheimer's disease, thereby making it possible to verify the effectiveness of the treatment for mild cognitive impairment and/or mild Alzheimer's disease which has been performed on the subject. When the therapeutic effect cannot be observed, it is possible to discontinue the treatment and to make another treatment plan. Therefore, the method for determining a therapeutic effect of the present invention can be used supplementarily in the judgment of the effectiveness of the treatment for mild cognitive impairment and/or mild Alzheimer's disease, and, in some cases, a doctor can finally judge whether the treatment is effective, in combination with any other finding. The method for determining a therapeutic effect of the present invention is also advantageous in that the method can suppress unnecessary dosing, thereby contributing to a reduction in medical cost and a reduction in burden on patients. The method for determining a therapeutic effect of the present invention further enables determination based on a biological sample collected by a less invasive method such as blood collection, and is thus advantageous also in that the method enables determination of the therapeutic effect on mild cognitive impairment and/or mild Alzheimer's disease at many facilities using biological samples obtained in a less invasive manner.

### <<Method for Determining Progress State of Pathological Condition>>

According to a third aspect of the present invention, a method for determining the progress state of the pathological condition of mild cognitive impairment and/or mild Alzheimer's disease is provided. According to the method for determining the progress state of the pathological condition of the present invention, it is possible to determine the degree of the progress state of the pathological condition of mild cognitive impairment and/or mild Alzheimer's disease using the amount or concentration of the biomolecule of the present invention in a biological sample from a test subject as an index. That is, the method for determining the progress state of the pathological condition of the present invention is characterized by correlating the amount or concentration of the biomolecule of the present invention with the progress state of the pathological condition of mild cognitive impairment and/or mild Alzheimer's disease in the test subject.

In the method for determining the progress state of the pathological condition of the present invention, as in the detection method of the present invention, the step (E) of measuring the amount or concentration of the biomolecule of the present invention in the biological sample from the test subject is carried out. When any other biomolecule is used as the index in addition to the biomolecule of the present invention, the step (Z) of measuring the amount or concentration of the other biomolecule in the biological sample from the test subject is carried out. The concentration of the biomolecule can be measured in the same manner as in the detection method of the present invention.

The method for determining the progress state of the pathological condition of the present invention can further include the step (F) of determining the degree of the progress state of the pathological condition of mild cognitive impairment and/or mild Alzheimer's disease for the test subject, using the amount or concentration of the biomolecule measured in step (E) as an index.

When the biomolecule of the present invention is the biomolecule (a1), the step (F) can be carried out by the step (F-a1-1) of comparing the amount or concentration of the biomolecule (a1) of the present invention in the biological sample from the test subject with a preliminarily measured amount or concentration of the biomolecule in the biological sample from the test subject; and the step (F-a1-2) of determining that the progress of the pathological condition is likely to have stopped or that the pathological condition is likely to have got well, when the amount or concentration of the biomolecule (a1) of the present invention in the biological sample from the test subject is equal to or lower than the preliminarily measured amount or concentration of the biomolecule.

Also, in the step (F-a1-2), when the amount or concentration of the biomolecule (a1) in the biological sample from the test subject is higher than the preliminarily measured amount or concentration of the biomolecule, it can be determined that the pathological condition is likely to have progressed.

When the biomolecule of the present invention is the biomolecule (b1), the step (F) can be carried out by the step (F-b1-1) of comparing the amount or concentration of the biomolecule (b1) of the present invention in the biological sample from the test subject with a preliminarily measured amount or concentration of the biomolecule in the biological sample from the test subject; and the step (F-b1-2) of determining that the progress of the pathological condition is likely to have stopped or that the pathological condition is likely to have got well, when the amount or concentration of the biomolecule (b1) of the present invention in the biological sample from the test subject is equal to or lower than the preliminarily measured amount or concentration of the biomolecule.

Also, in the step (F-a1-2), when the amount or concentration of the biomolecule (b1) in the biological sample from the test subject is higher than the preliminarily measured amount or concentration of the biomolecule, it can be determined that the pathological condition is likely to have progressed.

When two or more of the biomolecules of the present invention are used in combination for determination in the method for determining the progress state of the pathological condition of the present invention, the progress state of the pathological condition can be determined more accurately than when the biomolecule of the present invention is used singly for determination. When two or more of the biomolecules of the present invention are used in combination for determination in the method for determining the progress state of the pathological condition of the present invention, the step (E) and the step (F) can be performed on the respective biomolecules. In this case, the progress state of the pathological condition can be determined by combining the degrees of the progress state of the pathological condition indicated based on the individual biomolecules. For example, when the possibility that the pathological condition may have progressed is indicated for both of the two biomolecules of the present invention, the possibility that the pathological condition may have progressed is suggested more strongly than in the results obtained using each biomolecule singly. When the possibility that the pathological condition may have got well is indicated for both of the two biomolecules of the present invention, the possibility that the pathological condition may have got well is suggested more strongly than in the results obtained using each biomolecule singly.

The method for determining the progress state of the pathological condition of the present invention can further include the step (R) of determining the degree of the progress state of the pathological condition of mild cognitive impairment and/or mild Alzheimer's disease for the test subject, using the amount or concentration of the other biomolecule measured in step (Z) as an index. When the biomolecule of the present invention is the biomolecule (a1), the biomolecule (a2) and/or the biomolecule (b1) can be used as the other biomolecule(s). When the biomolecule of the present invention is the biomolecule (b1), the biomolecule (a2) can be used as the other biomolecule.

When the other biomolecule is the biomolecule (a2), the step (R) can be carried out by the step (R-a2-1) of comparing the amount or concentration of the other biomolecule in the biological sample from the test subject with a previously measured amount or concentration of the biomolecule in the biological sample from the test subject; and the step (R-a2-2) of:
determining that the progress of the pathological condition is likely to have stopped or that the pathological condition is likely to have got well, when the amount or concentration of any of TIMP4, NEFL, and TMPRSS15 among the biomolecules (a2) in the biological sample from the test subject is equal to or lower than the previously measured amount or concentration of the biomolecule in the biological sample from the test subject;
determining that the pathological condition is likely to have progressed, when the amount or concentration of any of TIMP4, NEFL, and TMPRSS15 among the biomolecules (a2) in the biological sample from the test subject is higher than the previously measured amount or concentration of the biomolecule in the biological sample from the test subject;
determining that the progress of the pathological condition is likely to have stopped or that the pathological condition is likely to have got well, when the amount or concentration of IGFBP1 among the biomolecules (a2) in the biological sample from the test subject is equal to or higher than the previously measured amount or concentration of the biomolecule in the biological sample from the test subject, and/or
determining that the pathological condition is likely to have progressed, when the amount or concentration of IGFBP1 among the biomolecules (a2) in the biological sample from the test subject is lower than the previously measured amount or concentration of the biomolecule in the biological sample from the test subject.

When the other biomolecule is the biomolecule (b1), the step (R) can be carried out by the step (R-b1-1) of comparing the amount or concentration of the other biomolecule in the biological sample from the test subject with a previously measured amount or concentration of the biomolecule in the biological sample from the test subject; and the step (R-b1-2) of:
determining that the progress of the pathological condition is likely to have stopped or that the pathological condition is likely to have got well, when the amount or concentration of the biomolecule (b1) in the biological sample from the test subject is equal to or lower than the previously measured amount or concentration of the biomolecule in the biological sample from the test subject, and/or
determining that the pathological condition is likely to have progressed, when the amount or concentration of the biomolecule (b1) in the biological sample from the test subject is higher than the previously measured amount or concentration of the biomolecule in the biological sample from the test subject.

When detection is performed using the other biomolecule in combination with the biomolecule of the present invention in the method for determining the progress state of the pathological condition of the present invention, the degree of the progress state of the pathological condition can be determined more accurately than when detection is performed using only the biomolecule of the present invention, as will be indicated in the Examples below. Therefore, the progress state of the pathological condition indicated in the step (R) can be used to supplement the progress state of the pathological condition indicated in the step (F). For example, when the possibility that the pathological condition may have progressed is indicated in the steps (F) and (R), the possibility that the pathological condition may have progressed is suggested more strongly than in the results obtained in the step (F) alone. When the possibility that the progress of the pathological condition may have stopped or the possibility that the pathological condition may have got well is indicated in the steps (F) and (R), the possibility that the progress of the pathological condition may have stopped or the possibility that the pathological condition may have got well is suggested more strongly than in the results obtained in the step (F) alone.

In the method for determining the progress state of the pathological condition of the present invention, the steps (E) and (F) or the steps (Z) and (R) can be carried out after a certain period of time has passed, in order to confirm the progress state of the pathological condition of mild cognitive impairment and/or mild Alzheimer's disease. The period of time until the next steps (E) and (F) or the next steps (Z) and (R) are carried out can be determined according to the expected therapeutic effect, when the test subject undergoes the treatment for mild cognitive impairment and/or mild Alzheimer's disease. For example, when the therapeutic effect is expected to be exerted about 1 to 2 months after start of the treatment, the steps (E) and (F) or the steps (Z) and (R) can be carried out every time a period of time of about one month or about two months has passed.

According to the method for determining the progress state of the pathological condition of the present invention, the progress state of the pathological condition of mild cognitive impairment and/or mild Alzheimer's disease can be confirmed or monitored in the subject who suffers from or is likely to suffer from mild cognitive impairment and/or mild Alzheimer's disease. Thus, when the test subject undergoes the treatment for mild cognitive impairment and/or mild Alzheimer's disease, it is possible to verify the effectiveness of the treatment for mild cognitive impairment and/or mild Alzheimer's disease performed on the subject. After completion of the treatment for mild cognitive impairment and/or mild Alzheimer's disease in the test subject, it is possible to verify the possibility of recurrence of the pathological condition of mild cognitive impairment and/or mild Alzheimer's disease. Therefore, the method for determining the progress state of the pathological condition of the present invention can be used supplementarily in the judgment of the progress state of the pathological condition of mild cognitive impairment and/or mild Alzheimer's disease, and, in some cases, a doctor can finally judge the progress state of the pathological condition, in combination with any other finding. The method for determining the progress state of the pathological condition of the present invention is also advantageous in that the method can suppress unnecessary dosing, thereby contributing to a reduction in medical cost and a reduction in burden on patients. The method for determining the progress state of the pathological condition of the present invention further enables determination based on a biological sample collected by a less invasive method such as blood collection, and is thus advantageous also in that the method enables determination of the progress state of the pathological condition of mild cognitive impairment and/or mild Alzheimer's disease at many facilities using biological samples obtained in a less invasive manner.

The method for determining the progress state of the pathological condition of the present invention can be used in combination with the detection method of the present invention. Specifically, the predetermined cutoff value is used as a boundary value for mild cognitive impairment and/or mild Alzheimer's disease to determine the likelihood of suffering and development risk of the test subject in the detection method of the present invention. On the other hand, in the method for determining the progress state of the pathological condition of the present invention, the amount or concentration of the biomolecule in the biological sample previously measured for the test subject is used for comparison. Hence, combination of the two methods advantageously make it possible to confirm both the progress state of the pathological condition in the test subject and the likelihood of suffering and the development risk.

### <<Biomarker>>

According to a fourth aspect of the present invention, there is provided use of the biomolecule of the present invention, as a biomarker for use in the detection or diagnosis of mild cognitive impairment and/or mild Alzheimer's disease and a biomarker for detection or diagnosis of mild cognitive impairment and/or mild Alzheimer's disease, each containing the biomolecule of the present invention. Also, according to the present invention, use of the biomolecule of the present invention as a biomarker in the method for detecting or diagnosing mild cognitive impairment and/or mild Alzheimer's disease is provided. The biomolecules of the present invention may be used singly, or two or more thereof may be used in combination. The biomolecule of the present invention may also be combined with any other biomolecule. In the present invention, the term “biomarker” refers to a living body-derived substance of which the presence and amount serve as an index of development of the disease and severity of its symptoms, and can be used as a marker for detecting, identifying, evaluating, etc. the disease. That is, according to the present invention, the biomolecule of the present invention can be used as a disease identification marker for mild cognitive impairment and/or mild Alzheimer's disease, and the biomolecule of the present invention can also be used to evaluate the severity of mild cognitive impairment and/or mild Alzheimer's disease.

### <<Screening Method>>

The method for determining a therapeutic effect of the present invention can be used to determine the effectiveness of a therapeutic agent or relieving agent for mild cognitive impairment and/or mild Alzheimer's disease, and thus a method for screening for a candidate for a therapeutic agent or relieving agent for mild cognitive impairment and/or mild Alzheimer's disease is also provided according to the present invention. Specifically, according to a fifth aspect of the present invention, a screening method in which the effectiveness of a therapeutic agent or relieving agent for mild cognitive impairment and/or mild Alzheimer's disease is determined using the amount or concentration of the biomolecule of the present invention in a biological sample from a test subject as an index. The screening method of the present invention is characterized by correlating the amount or concentration of the biomolecule of the present invention with the effectiveness of the candidate for a therapeutic agent or relieving agent on mild cognitive impairment and/or mild Alzheimer's disease.

In the screening method of the present invention, the step (G) of administering to a subject a candidate for a therapeutic agent or relieving agent for mild cognitive impairment and/or mild Alzheimer's disease is carried out, and then the step (H) of measuring the amount or concentration of the biomolecule of the present invention in a biological sample from the subject is carried out in the same manner as in the method for determining a therapeutic effect of the present invention. When any other biomolecule is used as the index in addition to the biomolecule of the present invention, the step (W) of measuring the amount or concentration of the other biomolecule in the biological sample from the subject is carried out. The concentration of the biomolecule can be measured in the same manner as in the detection method of the present invention.

The screening method of the present invention can further include the step (I) of determining the degree of a therapeutic effect of the candidate for a therapeutic agent or relieving agent on mild cognitive impairment and/or mild Alzheimer's disease, using the amount or concentration of the biomolecule of the present invention measured in step (H) as an index.

When the biomolecule of the present invention is the biomolecule (a1), the step (I) can be carried out by the step (I-a1-1) of comparing the amount or concentration of the biomolecule (a1) of the present invention in the biological sample from the subject with a predetermined cutoff value; and the step (I-a1-2) of determining that the candidate agent is likely to be effective as the therapeutic agent or relieving agent, when the amount or concentration of the biomolecule (a1) of the present invention in the biological sample from the subject is equal to or lower than the cutoff value or lower than the cutoff value.

Also, in the step (I-a1-2), when the amount or concentration of the biomolecule (a1) of the present invention in the biological sample from the test subject is equal to or higher than the cutoff value or higher than the cutoff value, it can be determined that the candidate agent is likely to be ineffective as the therapeutic agent or relieving agent (or the candidate agent is less likely to be effective as the therapeutic agent or relieving agent).

When the biomolecule of the present invention is the biomolecule (b1), the step (I) can be carried out by the step (I-b1-1) of comparing the amount or concentration of the biomolecule (b1) of the present invention in the biological sample from the subject with a predetermined cutoff value; and the step (I-b1-2) of determining that the candidate agent is likely to be effective as the therapeutic agent or relieving agent, when the amount or concentration of the biomolecule (b1) of the present invention in the biological sample from the subject is equal to or lower than the cutoff value or lower than the cutoff value.

Also, in the step (I-b1-2), when the amount or concentration of the biomolecule (b1) of the present invention in the biological sample from the test subject is equal to or higher than the cutoff value or higher than the cutoff value, it can be determined that the candidate agent is likely to be ineffective as the therapeutic agent or relieving agent (or the candidate agent is less likely to be effective as the therapeutic agent or relieving agent).

When two or more of the biomolecules of the present invention are used in combination for determination in the screening method of the present invention, the screening for the candidate agent can be performed more accurately than when the biomolecule of the present invention is used singly for determination, as will be indicated in the Examples below. When two or more of the biomolecules of the present invention are used in combination for screening in the screening method of the present invention, the step (H) and the step (I) can be performed on the respective biomolecules. In this case, the likelihood of effectiveness as the therapeutic agent or relieving agent can be determined more accurately by combining the degrees of the therapeutic effect indicated based on the individual biomolecules. For example, when the likelihood that the candidate agent may be effective as the therapeutic agent or relieving agent is indicated for both of the two biomolecules of the present invention, the likelihood of effectiveness as the therapeutic agent or relieving agent is suggested more strongly than in the results obtained using each biomolecule singly. When the likelihood that the candidate agent may be ineffective as the therapeutic agent or relieving agent is indicated (or it is indicated that the candidate agent is less likely to be effective as the therapeutic agent or relieving agent) for both of the two biomolecules of the present invention, the likelihood of effectiveness as the therapeutic agent or relieving agent is denied more strongly than in the results obtained using each biomolecule singly.

The screening method of the present invention can further include the step (S) of determining the degree of the therapeutic effect on mild cognitive impairment and/or mild Alzheimer's disease for the subject who has undergone the treatment, using the amount or concentration of the other biomolecule measured in step (W) as an index. When the biomolecule of the present invention is the biomolecule (a1), the biomolecule (a2) and/or the biomolecule (b1) can be used as the other biomolecule(s). When the biomolecule of the present invention is the biomolecule (b1), the biomolecule (a2) can be used as the other biomolecule.

When the other biomolecule is the biomolecule (a2), the step (S) can be carried out by the step (S-a2-1) of comparing the amount or concentration of the biomolecule (a2) in the biological sample from the test subject with a predetermined cutoff value; and the step (S-a2-2) of determining that the likelihood that the candidate agent is likely to be effective as the therapeutic agent or relieving agent when the amount or concentration of any of TIMP4, NEFL and TMPRSS15 among the biomolecules (a2) in the biological sample from the test subject is equal to or lower than the cutoff value or lower than the cutoff value, and determining that the likelihood that the candidate agent is likely to be effective as the therapeutic agent or relieving agent when the amount or concentration of IGFBP1 among the biomolecules (a2) in the biological sample from the test subject is equal to or higher than the cutoff value or higher than the cutoff value.

In the step (S-a2-2), it is determined that the likelihood that the candidate agent may be ineffective as the therapeutic agent or relieving agent is indicated (or the candidate agent is less likely to be effective as the therapeutic agent or relieving agent) when the amount or concentration of any of TIMP4, NEFL and TMPRSS15 among the biomolecules (a2) in the biological sample from the test subject is equal to or higher than the cutoff value or higher than the cutoff value, and that the treatment is likely to be ineffective (or the treatment is less likely to be effective) when the amount or concentration of IGFBP1 among the biomolecules (a2) in the biological sample from the test subject is equal to or lower than the cutoff value or lower than the cutoff value.

When the other biomolecule of the present invention is the biomolecule (b1), the step (S) can be carried out by the step (S-b1-1) of comparing the amount or concentration of the biomolecule (b1) of the present invention in the biological sample from the test subject with a predetermined cutoff value; and the step (S-b1-2) of determining that the candidate agent is likely to be effective as the therapeutic agent or relieving agent, when the amount or concentration of the biomolecule (b1) of the present invention in the biological sample from the test subject is equal to or lower than the cutoff value or lower than the cutoff value.

Also, in the step (S-b1-2), when the amount or concentration of the biomolecule (b1) in the biological sample from the test subject is equal to or higher than the cutoff value or higher than the cutoff value, it can be determined that the candidate agent is likely to be ineffective as the therapeutic agent or relieving agent (or the candidate agent is less likely to be effective as the therapeutic agent or relieving agent).

When detection is performed using the other biomolecule in combination with the biomolecule of the present invention in the screening method of the present invention, the likelihood of effectiveness of the therapeutic agent or relieving agent can be determined more accurately than when detection is performed using only the biomolecule of the present invention, as will be indicated in the Examples below. Therefore, the determination results indicated in the step (S) can be used to supplement the determination results indicated in the step (I). For example, when the likelihood that the candidate agent may be effective as the therapeutic agent or relieving agent is indicated in the steps (I) and (S), the likelihood of effectiveness of the therapeutic agent or relieving agent is suggested more strongly than in the results obtained in the step (I) alone. When the likelihood that the candidate agent may be ineffective as the therapeutic agent or relieving agent is indicated (or it is indicated that the candidate agent is less likely to be effective as the therapeutic agent or relieving agent) in the steps (I) and (S), the likelihood of effectiveness of the therapeutic agent or relieving agent is denied more strongly than in the results obtained in the step (I) alone.

The cutoff value used in the screening method of the present invention can be set according to the descriptions about the detection method of the present invention. In addition to this, measured values (reference values) of the amounts or concentrations of the biomolecule of the present invention and the other biomolecule in the biological sample from the test subject before the administration can be used in place of the cutoff value in the screening method of the present invention. In this case, the screening method of the present invention may further include the step of measuring the amount or concentration of the biomolecule of the present invention and/or the other biomolecule in the biological sample from the subject, before administration of the therapeutic agent or relieving agent for mild cognitive impairment and/or mild Alzheimer's disease or after administration thereof (preferably soon after administration, e.g., within one to two days, within a few days, or within one week). In the case where the biomolecule is any of FGF-19, PLA2G10, CPA2, TIMP4, NEFL and TMPRSS15, it can be determined that the candidate agent is likely to be effective as the therapeutic agent or relieving agent when the amount or concentration of the biomolecule in the biological sample from the test subject is lower than the reference value. In the case where the biomolecule is IGFBP1, it can be determined that the candidate agent is likely to be effective as the therapeutic agent or relieving agent when the amount or concentration of the biomolecule in the biological sample from the test subject is higher than the reference value. Also, in the case where the biomolecule is biomolecule (b1), it can be determined that the candidate agent is likely to be effective as the therapeutic agent or relieving agent when the amount or concentration of the biomolecule in the biological sample from the test subject is lower than the reference value.

In the screening method of the present invention, the steps (H) and (I) or the steps (W) and (S) can be carried out after a certain period of time has passed after administration of the candidate for the therapeutic agent or relieving agent for mild cognitive impairment and/or mild Alzheimer's disease, or after a certain period of time has passed after the implementation of the administration. The period of time from the start of the administration to the implementation of the steps (H) and (I) or the steps (W) and (S) can be determined according to the expected therapeutic effect. For example, when the therapeutic effect is expected to be exerted about 1 to 2 months after start of the administration, the steps (H) and (I) or the steps (W) and (S) can be carried out about one month or about two months after start of the administration, and, subsequently, the steps (H) and (I) or the steps (W) and (S) may be carried out again after a period of time of about one month or about two months.

Since the screening method of the present invention is intended to determine the therapeutic effect, the subject to whom the candidate for the therapeutic agent or relieving agent is administered is a subject suffering from mild cognitive impairment and/or mild Alzheimer's disease, dementia with Lewy bodies, and frontotemporal lobar degeneration. In the present invention, the “subject suffering from mild cognitive impairment and/or mild Alzheimer's disease” is, for example, a subject diagnosed by a doctor as developing mild cognitive impairment and/or mild Alzheimer's disease, and may be a subject whose results obtained by other examination methods suggest the possibility that the subject may suffer from mild cognitive impairment and/or mild Alzheimer's disease. The same applies to the subjects suffering from a disease other than mild cognitive impairment and/or mild Alzheimer's disease.

The candidate for the therapeutic agent or relieving agent to be screened by the screening method of the present invention is not limited, and examples of the relieving agent for mild cognitive impairment and/or mild Alzheimer's disease include foods, quasi-drugs (e.g. medical cosmetics), feeds (e.g. pet foods), cosmetics and skin care products which have the function of relieving symptoms of mild cognitive impairment and/or mild Alzheimer's disease. The foods include supplements, foods for specified health use, and foods with functional claims. In addition, the meaning of the term “relieving” include amelioration.

In the step (I) of the screening method of the present invention, for example, when the amount or concentration of the biomolecule (a1) of the present invention in the biological sample from the subject who has undergone the administration is about 1.3 times or less, about 1.2 times or less, about 1.1 times or less, or about 1.05 times or less the average value of the amount or concentration of the biomolecule for the normal subject group, it can be determined that the candidate agent is likely to be effective as the therapeutic agent or relieving agent. Also, in the step (I) of the screening method of the present invention, when the amount or concentration of the biomolecule (a1) of the present invention in the biological sample from the subject who has undergone the administration is lower than the average value of the amount or concentration of the biomolecule for the affected subject group, or is about 0.95 times or less, about 0.9 times or less, about 0.8 times or less, or about 0.7 times or less the average value, it can be determined that the candidate agent is likely to be effective as the therapeutic agent or relieving agent.

Also, in the step (I) of the method for determining a therapeutic effect of the present invention, when the amount or concentration of the biomolecule (b1) of the present invention in the biological sample from the subject who has undergone the administration is about 1.3 times or less, about 1.2 times or less, about 1.1 times or less, or about 1.05 times or less the average value of the amount or concentration of the biomolecule for the normal subject group, it can be determined that the candidate agent is likely to be effective as the therapeutic agent or relieving agent. Also, in the step (I) of the method for determining a therapeutic effect of the present invention, when the amount or concentration of the biomolecule (b1) of the present invention in the biological sample from the subject who has undergone the administration is lower than the average value of the amount or concentration of the biomolecule for the affected subject group, or is about 0.95 times or less, about 0.9 times or less, about 0.8 times or less, or about 0.7 times or less the average value, it can be determined that the candidate agent is likely to be effective as the therapeutic agent or relieving agent.

### <<Detection Kit>>

According to a sixth aspect of the present invention, a kit for detection or diagnosis of mild cognitive impairment and/or mild Alzheimer's disease, comprising a means of quantifying the biomolecule of the present invention in a biological sample from a subject is provided. The kit of the present invention may include a means of quantifying any other biomolecule (for example, biomolecule (a2)) in addition to the means of quantifying the biomolecule of the present invention. The kit of the present invention is typically a kit for detecting or determining mild cognitive impairment and/or mild Alzheimer's disease according to the detection method of the present invention. The means of quantifying the biomolecule of the present invention includes, for example, substances that specifically bind to the biomolecule, and is typically an antibody, an aptamer, and a drug against the biomolecule. The means of quantifying the biomolecule also includes mass spectrometers for use in the mass spectrometry methods as described above.

In the kit of the present invention, when the means of quantifying the biomolecule of the present invention is an antibody, the kit of the present invention includes a reagent (and optionally a device) required to measure the concentration of biomolecules by an immunoassay utilizing the antibody. The kit of the present invention is, for example, a kit for measuring the concentration of the biomolecule by a sandwich method, and the kit may include a microtiter plate, an anti-biomolecule antibody for capture, an alkaline phosphatase- or peroxidase-labeled anti-biomolecule antibody, and an alkaline phosphatase substrate or peroxidase substrate. The kit of the present invention is, for example, a kit for measuring the concentration of the biomolecule by a sandwich method utilizing a secondary antibody, and the kit may include a microtiter plate, an anti-biomolecule antibody for capture, an anti-biomolecule antibody as a primary antibody, an antibody for an alkaline phosphatase- or peroxidase-labeled anti-biomolecule antibody, and an alkaline phosphatase substrate or peroxidase substrate.

Further, the kit of the present invention is, for example, a kit for measuring the concentration of the biomolecule by immunochromatography, and the kit can be configured in such a manner that an antibody storage section in which a first anti-biomolecule antibody labeled with colloidal gold or the like is stored and a determination section in which a second anti-biomolecule antibody (preferably an antibody that recognizes another epitope of the biomolecule) is fixed in a line on a cellulose film or the like are connected by a narrow groove.

In the kit of the present invention, when the means of quantifying the biomolecule is a mass spectrometer, the kit of the present invention optionally includes an internal standard device in addition to the mass spectrometer. The use of an internal standard can correct the extraction efficiency and ionization efficiency for each analysis at the time of measurement with the mass spectrometer. The internal standard used in mass spectrometry includes deuterated biomolecules.

In addition to the above, the kit of the present invention can be implemented according to the descriptions about the detection method and determination methods of the present invention.

### <<Treatment Method>>

Treatment for mild cognitive impairment and/or mild Alzheimer's disease can be performed on a subject specified or diagnosed as being in need of the treatment for mild cognitive impairment and/or mild Alzheimer's disease by the detection method of the present invention or the diagnosis method of the present invention. Thus, according to a seventh aspect of the present invention, there is provided a method for treating mild cognitive impairment and/or mild Alzheimer's disease, comprising: the step (A) of measuring the amount or concentration of the biomolecule of the present invention in a biological sample from a subject; the step (B) of determining the likelihood of suffering from mild cognitive impairment and/or mild Alzheimer's disease for the test subject from whom the biological sample has been collected using the amount or concentration of the biomolecule of the present invention measured in the step (A); and the step (J) of performing treatment for mild cognitive impairment and/or mild Alzheimer's disease on a subject determined to suffer from (or to be likely to suffer from) mild cognitive impairment and/or mild Alzheimer's disease. In the treatment method of the present invention, the step of measuring the amount or concentration of the biomolecule and the step of determining mild cognitive impairment and/or mild Alzheimer's disease can be carried out according to the descriptions about the detection method of the present invention and the diagnosis method of the present invention (that is, the steps (A), (B), (B-a1-1), (B-a1-2), (B-b1-1) and (B-b1-2) as well as the steps (X), (P), (P-a1-1), (P-a1-2), (P-b1-1) and (P-b1-2)). Also, the treatment for mild cognitive impairment and/or mild Alzheimer's disease can be performed according to the descriptions about the method for determining a therapeutic effect of the present invention.

### EXAMPLES

Hereinafter, the present invention will be described in more detail by way of the following examples, but is not limited thereto.

### Example 1: Screening for Biomarker (Protein) of Mild Cognitive Impairment and Mild Alzheimer's Disease

### (1) Preparation of Specimen

Proteomics was performed on specimens (352 specimens) from the National Center for Geriatrics and Gerontology (NCGG) biobank (blood samples obtained from residents of Aichi prefecture and its surroundings who agreed to this study). The specimens were classified into the following four groups (hereafter, the four groups are collectively referred to as “specimen group A”). Specifically, the specimens were classified into: normal cognitive function (number of specimens: 103, Normal Cognitive, sometimes referred to herein as “NC”); stable mild cognitive impairment (number of specimens: 62, stable Mild Cognitive Impairment, sometimes referred to herein as “sMCI”); progressive mild cognitive impairment (number of specimens: 125, progressive Mild Cognitive Impairment, sometimes referred to herein as “pMCI”); and mild Alzheimer's disease (number of specimens: 62, Mild Alzheimer's disease, sometimes referred to herein as “AD”), and proteomics was performed on plasma specimens obtained by blood collection from the following subjects. Here, NC is a subject who was confirmed, through doctor's diagnosis, to have normal cognitive function multiple times after blood collection. sMCI is a subject who was diagnosed as having MCI by a doctor at the time of blood collection, maintained MCI for 3 years or more after blood collection, and had not yet been confirmed in terms of transition to AD. pMCI is a subject who was diagnosed as having MCI by a doctor at the time of blood collection, and transitioned to MCI within 5 years after blood collection (sMCI and pMCI overlap in a period of from 3 to 5 years after blood collection). AD is a subject who was confirmed to have AD through doctor's diagnosis at the time of blood collection. As for the breakdown of the AD group, the result of a mini-mental state examination (MMSE examination, dementia screening examination) at the time of blood collection was 23 for more than half of the subjects, and 22 for the remaining subjects.

In addition to the above, proteomics was performed on specimens (352 specimens) from the National Center for Geriatrics and Gerontology (NCGG) biobank (blood samples obtained from residents of Aichi prefecture and its surroundings who agreed to this study). The specimens were classified into the following three groups (hereafter, the three groups are collectively referred to as “specimen group B”). Specifically, the specimens were classified into: normal cognitive function (number of specimens: 103, NC); mild cognitive impairment (number of specimens: 187, Mild Cognitive Impairment, sometimes referred to herein as “MCI”); and mild Alzheimer's disease (number of specimens: 62, AD), and proteomics was performed on plasma specimens obtained by blood collection from the following subjects. Here, NC is a subject who was confirmed, through doctor's diagnosis, to have normal cognitive function multiple times after blood collection. MCI is a subject who satisfies the requirements: of 1) having memory impairment that cannot be explained to be caused merely by the influence of age or educational level; that 2) complaints of forgetfulness are expressed from the subject himself/herself or his/her family members; of 3) having a general cognitive function within the normal range; of 4) being independent in activities of daily living; and of 5) having no dementia (source: Mild Cognitive Impairment in e-Health Net, Ministry of Health, Labor and Welfare). AD is a subject who was confirmed to have AD through doctor's diagnosis at the time of blood collection. As for the breakdown of the AD group, the result of a mini-mental state examination (MMSE examination, dementia screening examination) at the time of blood collection was 23 for more than half of the subjects, and 22 for the remaining subjects.

### (2) Proteomics

Plasma proteomics of 368 proteins was performed on the specimen group A in the above (1), using Olink Proteomics Multiplex-Inflammation Kit (Olink), Neurology Kit (Olink) and Neuro-Exploratory Kit (Olink). Plasma proteomics of 602 proteins was also performed on the specimen group B in the above (1), using the above kit. In the measurement, two oligonucleotide-attached antibodies recognizing different epitopes were used for one target protein. Only when two antibodies bind to one target protein molecule, the corresponding oligonucleotides form a hybrid, and the resulting double-stranded nucleic acid is quantified by high-throughput real-time PCR, thereby making it possible to quantify the target protein.

The protein was quantified according to the following procedures.

a. Antigen Antibody Reaction

Plasma and a reaction solution containing oligonucleotide-attached antibodies against the protein to be detected were mixed and allowed to react at 4° C. for 16 hours.

b. Elongation/Pre-Amplification Reaction

The oligonucleotides of the antibodies which bound to the protein were hybridized, and an elongation reaction was performed using PCR to form an amplicon.

c. Detection Reaction

Quantitative PCR was performed using a high-throughput real-time PCR device (Biomark, Fluidigm).

d. Analysis

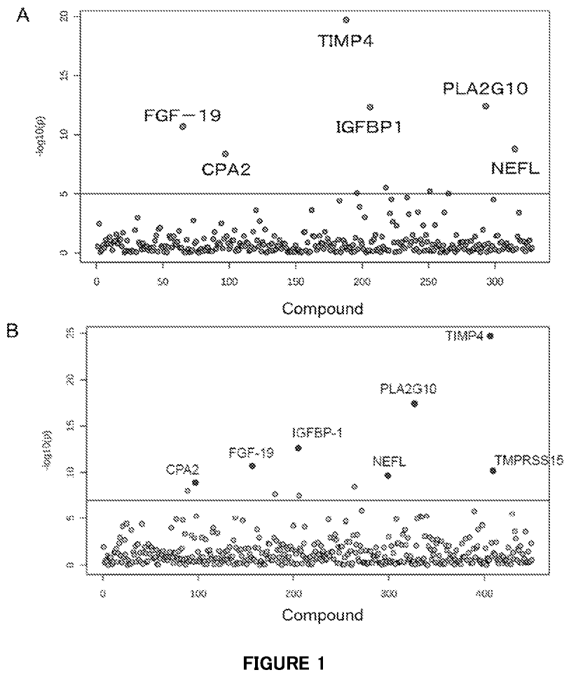

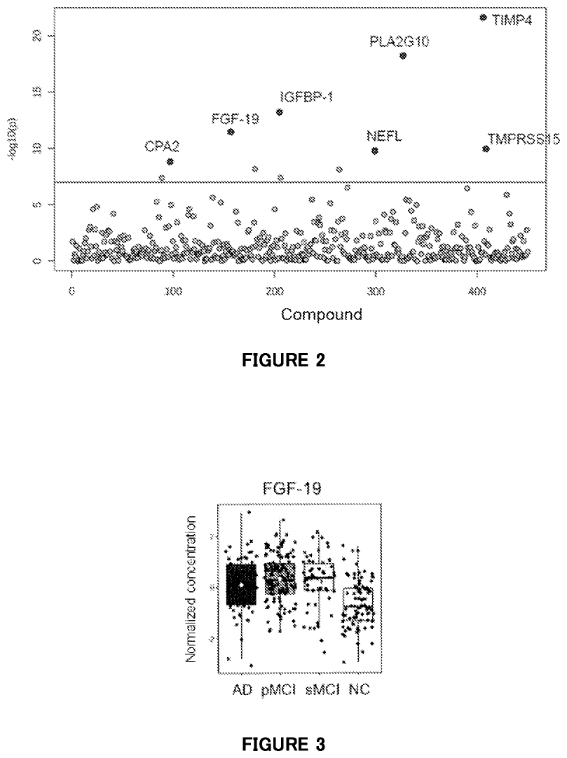

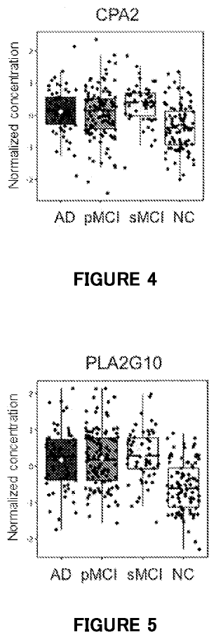

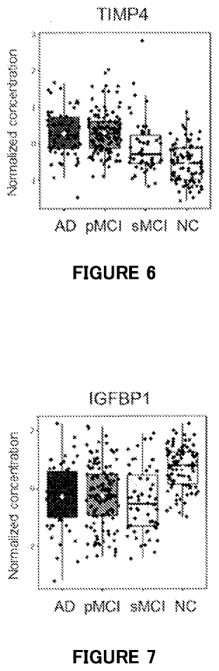

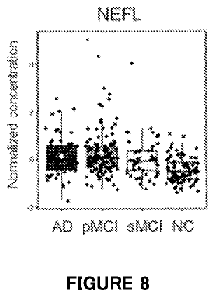

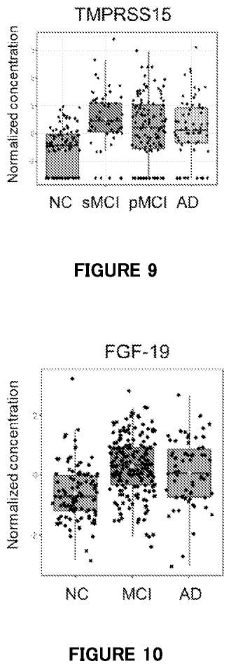

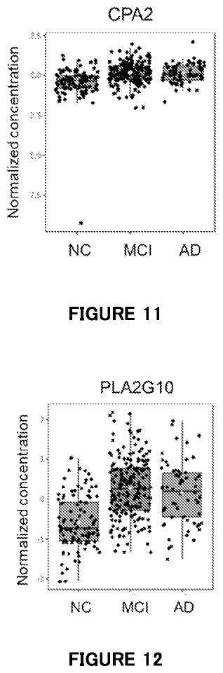

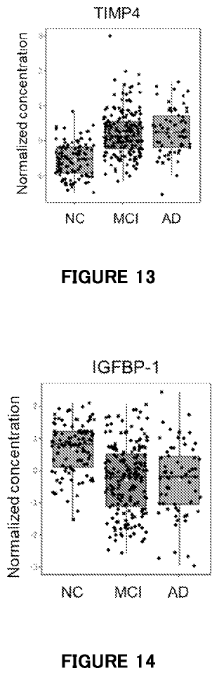

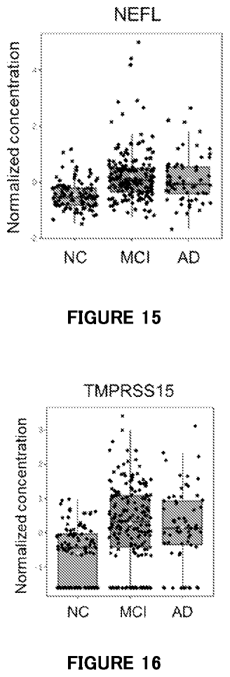

Olink NPX Manager (analysis software, Olink) was used to obtain relative quantification values of the proteins as normalized values on a log 2 scale. The values were saved as data in CSV format, and FIGS. 1 and 2 were created using the ANOVA tool of Statistical Analysis (https://www.metaboanalyst.ca/MetaboAnalyst/upload/StatUploadView.xhtml) using MetaboAnalyst (https://www.metaboanalyst.ca/). FIGS. 3 to 8 were obtained by clicking on a target spot for ANOVA in FIG. 1A using MetaboAnalyst (https://www.metaboanalyst.ca/); FIG. 9 was obtained by clicking on a target spot for ANOVA in FIG. 1B using MetaboAnalyst (https://www.metaboanalyst.ca/); and FIGS. 10 to 16 were obtained by clicking on a target spot for ANOVA in FIG. 2 using MetaboAnalyst (https://www.metaboanalyst.ca/).

### (3) Results

The results were as shown in FIGS. 1 to 16.

FIG. 1A shows relative quantification values of 368 proteins in the plasma for the sMCI, pMCI, and AD groups to the quantification values for the NC group; FIG. 1B shows relative quantification values of 602 proteins in the plasma of the sMCI, pMCI, and AD groups to the quantification values for the NC group; and FIG. 2 shows relative quantification values of 602 proteins in the plasma of the MCI and AD groups to the quantification values for the NC group. From FIG. 1A, the sMCI, pMCI, and AD groups were confirmed to have significant differences in relative quantification values of six proteins (FGF-19, PLA2G10, CPA2, TIMP4, IGFBP1, and NEFL), as compared with the NC group (p<0.00001, One-way ANOVA test). From FIG. 1B, the sMCI, pMCI, and AD groups were confirmed to have significant differences in relative quantification value of TMPRSS15 in addition to the six proteins (FGF-19, PLA2G10, CPA2, TIMP4, IGFBP1, and NEFL) in FIG. 1A, as compared with the NC group (p<0.0000001, One-way ANOVA test). From FIG. 2, the MCI and AD groups were confirmed to have significant differences in relative quantification values of seven proteins (FGF-19, PLA2G10, CPA2, TIMP4, IGFBP1, NEFL, and TMPRSS15), as compared with the NC group (p<0.00001, One-way ANOVA test).

FIGS. 3 to 9 show the relative quantification values of the NC, sMCI, pMCI and AD groups for the seven proteins for which significant differences were confirmed in FIG. 1. FIGS. 10 to 16 show the relative quantification values of the NC, MCI and AD groups for the seven proteins for which significant differences were confirmed in FIG. 2. The results of FIGS. 3 to 16 showed that it is possible to detect NC and MCI (sMCI and pMCI) separately from each other and to separate and to detect NC and AD separately from each other by using the expression level of each of the seven proteins as the index. In addition, it was confirmed that, as compared with the NC group, the MCI groups (sMCI group and pMCI group) and the AD group were significantly high in expression levels of FGF-19, PLA2G10, CPA2, TIMP4, NEFL and TMPRSS15, and significantly low in expression level of IGFBP1. It has been reported that TIMP4 increases in the AD group (J Alzheimers Dis., 45(1): 245-52, 2015); that IGFBP1 fluctuates in the AD group (J Endocrinol Invest., 24(3): 139-46, 2001); that NEFL increases in the AD group (Lancet Neurol., 19(6): 513-521, 2020, Scientific Reports, Vol. 8, No. 17368, 2018); and that TMPRSS15 is associated with Alzheimer's disease (Nat Rev Neurosci., 16(9): 564-74, 2015, Int J Mol Sci., 22(17):9120, 2021).

### Example 2: Discrimination by Mild Cognitive Impairment and Mild Alzheimer's Disease Marker (Protein)

### (1) ROC Analysis

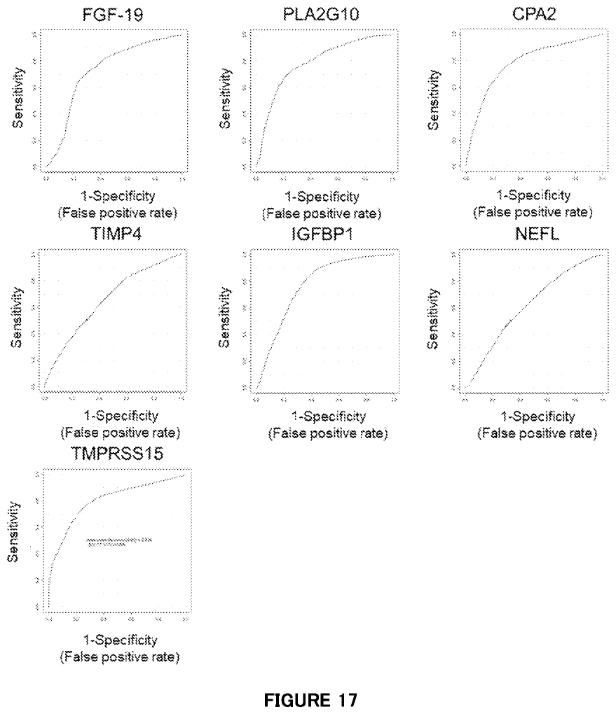

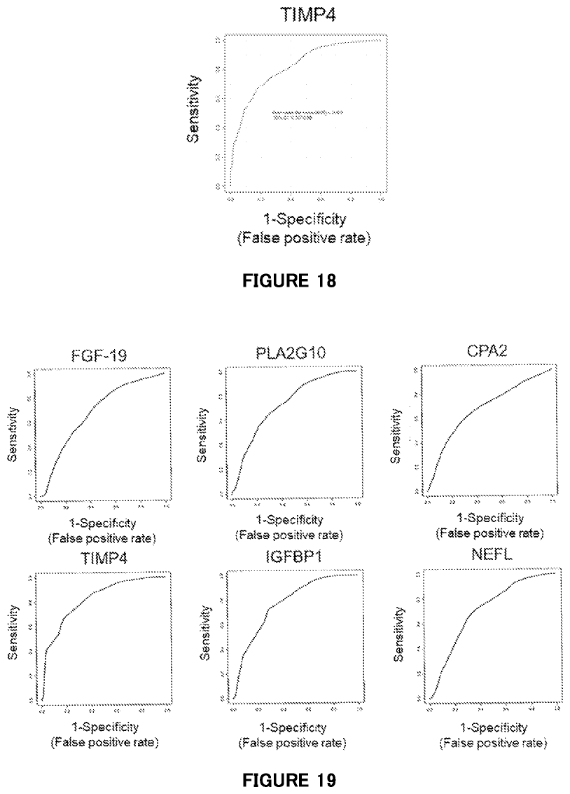

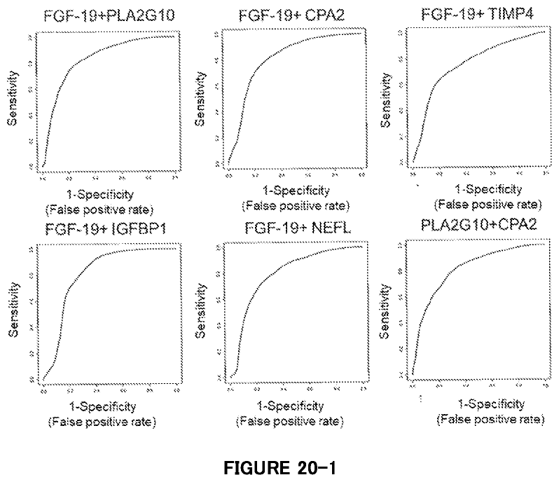

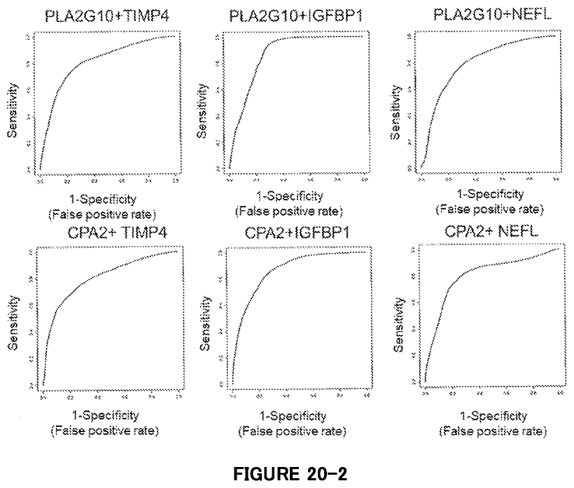

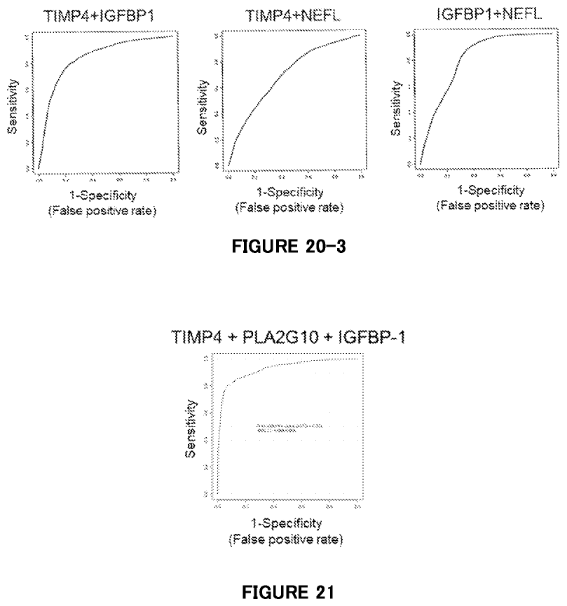

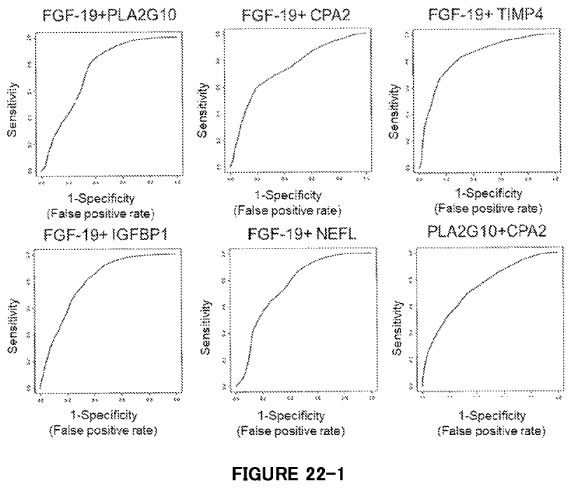

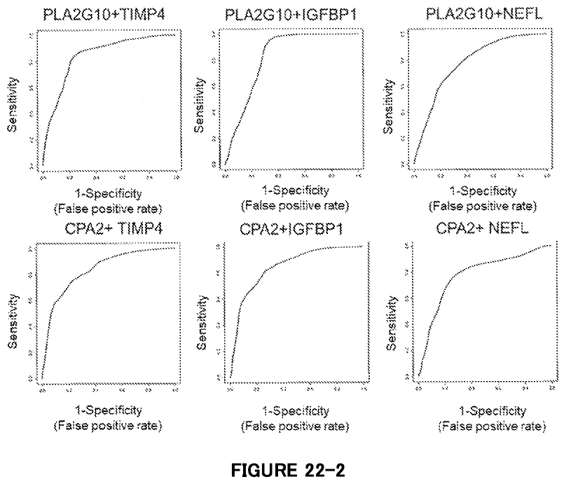

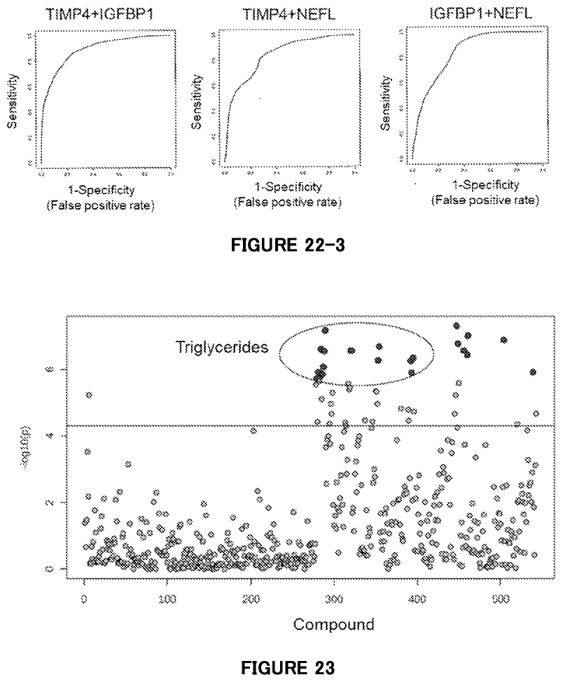

For the seven proteins for which significant differences were confirmed in Example 1, discrimination between NC and sMCI, discrimination between NC and MCI, and discrimination between NC and AD were analyzed using ROC curves (Tables 2 to 4 and FIGS. 17 to 19). Also, for combinations of the seven proteins, discrimination between NC and sMCI, discrimination between NC and MCI, and discrimination between NC and AD were analyzed using ROC curves (Tables 5 to 7 and FIGS. 20 to 22). These analyses were performed using the statistical software MetaboAnalyst (https://www.metaboanalyst.ca/MetaboAnalyst/upload/RocUploadView.xhtml) (Xia Laboratory, McGill University).

### (2) Results

The results were as shown in Tables 2 to 7 and FIGS. 17 to 22.

TABLE 2
Example of discrimination between NC and
sMCI by single biomarker (protein)

| Protein | AUC | 95% CI |
|---|---|---|
| FGF-19 | 0.731 | 0.634-0.846 |
| PLA2G10 | 0.798 | 0.708-0.877 |
| CPA2 | 0.794 | 0.711-0.872 |
| TIMP4 | 0.668 | 0.544-0.773 |
| IGFBP1 | 0.782 | 0.651-0.864 |
| NEFL | 0.629 | 0.28-0.764 |
| TMPRSS15 | 0.819 | 0.751-1.886 |

TABLE 3
Example of discrimination between NC
and MCI by single biomarker (protein)

| Protein | AUC | 95% CI |
|---|---|---|
| FGF-19 | 0.761 | 0.704-0.837 |
| PLA2G10 | 0.796 | 0.726-0.857 |
| CPA2 | 0.741 | 0.622-0.817 |
| TIMP4 | 0.827 | 0,767-0.88 |
| IGFBP1 | 0.763 | 0.708-0.824 |
| NEFL | 0.768 | 0.707-0.821 |
| TMPRSS15 | 0.739 | 0.684-0.8 |

TABLE 4
Example of discrimination between NC
and AD by single biomarker (protein)

| Protein | AUC | 95% CI |
|---|---|---|
| FGF-19 | 0.695 | 0.364-0.805 |
| PLA2G10 | 0.746 | 0.657-0.84 |
| CPA2 | 0.683 | 0.556-0.777 |
| TIMP4 | 0.829 | 0.755-0.906 |
| IGFBP1 | 0.783 | 0.675-0.884 |
| NEFL | 0.719 | 0.602-0.824 |

TABLE 5
Example of discrimination between NC and sMCI
by combination of biomarkers (proteins)

| Combination No. | Combination | AUC | 95% CI |
|---|---|---|---|
| 001 | FGF-19 and PLA2G10 | 0.838 | 0.754-0.92 |
| 002 | FGF-19 and CPA2 | 0.82 | 0.73-0.917 |
| 003 | FGF-19 and TIMP4 | 0.769 | 0.667-0.866 |
| 004 | FGF-19 and IGFBP1 | 0.818 | 0.726-0.913 |
| 005 | FGF-19 and NEFL | 0.804 | 0.717-0.903 |
| 006 | PLA2G10 and CPA2 | 0.824 | 0.745-0.903 |
| 007 | PLA2G10 and TIMP4 | 0.819 | 0.724-0.891 |
| 008 | PLA2G10 and IGFBP1 | 0.875 | 0.798-0.941 |
| 009 | PLA2G10 and NEFL | 0.807 | 0.713-0.881 |
| 010 | PLA2G10 and TMPRSS15 | 0.869 | 0.812-0.914 |
| 011 | CPA2 and TIMP4 | 0.816 | 0.731-0.888 |
| 012 | CPA2 and IGFBP1 | 0.872 | 0.791-0.936 |
| 013 | CPA2 and NEFL | 0.814 | 0.73-0.893 |
| 014 | CPA2 and TMPRSS15 | 0.871 | 0.802-0.934 |
| 015 | TIMP4 and IGFBP1 | 0.852 | 0.752-0.932 |
| 016 | TIMP4 and NEFL | 0.716 | 0.599-0.812 |
| 017 | IGFBP1 and NEFL | 0.822 | 0.731-0.925 |
| 018 | PLA2G10, CPA2 and | 0.882 | 0.826-0.929 |
| | TMPRSS15 | | |
| 019 | PLA2G10, IGFBP1 and | 0.902 | 0.84-0.961 |
| | TMPRSS15 | | |
| 020 | CPA2, IGFBP1 and | 0.894 | 0.827-0.964 |
| | TMPRSS15 | | |

TABLE 6
Example of discrimination between NC and MCI
by combination of biomarkers (proteins)

| Combination No. | Combination | AUC | 95% CI |
|---|---|---|---|
| 021 | FGF-19 and PLA2G10 | 0.824 | 0.775-0.869 |
| 022 | FGF-19 and CPA2 | 0.802 | 0.75-0.848 |
| 023 | FGF-19 and TIMP4 | 0.855 | 0.797-0.898 |
| 024 | FGF-19 and IGFBP1 | 0.828 | 0.779-0.87 |
| 025 | FGF-19 and NEFL | 0.848 | 0.8-0.886 |
| 026 | FGF-19 and TMPRSS15 | 0.796 | 0.74-0.854 |
| 027 | PLA2G10 and CPA2 | 0.805 | 0.738-0.865 |
| 028 | PLA2G10 and TIMP4 | 0.877 | 0.82-0.91 |
| 029 | PLA2G10 and IGFBP1 | 0.884 | 0.85-0.915 |
| 030 | PLA2G10 and NEFL | 0.836 | 0.777-0.888 |
| 031 | PLA2G10 and TMPRSS15 | 0.826 | 0.766-0.882 |
| 032 | CPA2 and TIMP4 | 0.86 | 0.812-0.9 |
| 033 | CPA2 and IGFBP1 | 0.832 | 0.781-0.876 |
| 034 | CPA2 and NEFL | 0.827 | 0.766-0.874 |
| 035 | CPA2 and TMPRSS15 | 0.778 | 0.714-0.826 |
| 036 | TIMP4 and IGFBP1 | 0.896 | 0.857-0.935 |
| 037 | TIMP4 and NEFL | 0.857 | 0.805-0.898 |
| 038 | TIMP4 and TMPRSS15 | 0.888 | 0.847-0.927 |
| 039 | IGFBP1 and NEFL | 0.873 | 0.835-0.915 |
| 040 | IGFBP1 and TMPRSS15 | 0.799 | 0.754-0.849 |
| 041 | FGF-19, PLA2G10 and TIMP4 | 0.886 | 0.841-0.934 |
| 042 | FGF-19, PLA2G10 and NEFL | 0.867 | 0.819-0.903 |
| 043 | FGF-19, CPA2 and TIMP4 | 0.875 | 0.833-0.913 |
| 044 | FGF-19, CPA2 and IGFBP1 | 0.868 | 0.83-0.905 |
| 045 | FGF-19, CPA2 and NEFL | 0.861 | 0.813-0.907 |
| 046 | FGF-19, TIMP4 and IGFBP1 | 0.907 | 0.871-0.941 |
| 047 | FGF-19, TIMP4 and NEFL | 0.882 | 0.841-0.917 |
| 048 | FGF-19, IGFBP1 and NEFL | 0.906 | 0.879-0.941 |
| 049 | PLA2G10, CPA2 and TIMP4 | 0.883 | 0.836-0.919 |
| 050 | PLA2G10, CPA2 and NEFL | 0.842 | 0.78-0.892 |
| 051 | PLA2G10, TIMP4 and IGFBP1 | 0.931 | 0.899-0.956 |
| 052 | PLA2G10, TIMP4 and NEFL | 0.894 | 0.855-0.932 |
| 053 | PLA2G10, IGFBP1 and NEFL | 0.91 | 0.88-0.938 |
| 054 | CPA2, TIMP4 and IGFBP1 | 0.916 | 0.89-0.948 |
| 055 | CPA2, TIMP4 and NEFL | 0.883 | 0.836-0.923 |
| 056 | TIMP4, IGFBP1 and NEFL | 0.918 | 0.885-0.954 |

TABLE 7
Example of discrimination between NC and AD
by combination of biomarkers (proteins)

| Combination No. | Combination | AUC | 95% CI |
|---|---|---|---|
| 057 | FGF-19 and PLA2G10 | 0.766 | 0.665-0.871 |
| 058 | FGF-19 and CPA2 | 0.753 | 0.645-0.86 |
| 059 | FGF-19 and TIMP4 | 0.835 | 0.754-0.91 |
| 060 | FGF-19 and IGFBP1 | 0.807 | 0.71-0.883 |
| 061 | FGF-19 and NEFL | 0.766 | 0.647-0.857 |
| 062 | PLA2G10 and CPA2 | 0.76 | 0.674-0.867 |
| 063 | PLA2G10 and TIMP4 | 0.854 | 0.777-0.93 |
| 064 | PLA2G10 and IGFBP1 | 0.833 | 0.736-0.928 |
| 065 | PLA2G10 and NEFL | 0.788 | 0.687-0.881 |
| 066 | CPA2 and TIMP4 | 0.843 | 0.757-0.923 |
| 067 | CPA2 and IGFBP1 | 0.852 | 0.773-0.925 |
| 068 | CPA2 and NEFL | 0.786 | 0.685-0.882 |
| 069 | TIMP4 and IGFBP1 | 0.898 | 0.844-0.947 |
| 070 | TIMP4 and NEFL | 0.849 | 0.755-0.926 |
| 071 | IGFBP1 and NEFL | 0.851 | 0.763-0.935 |

From the results in Tables 2 to 4, it was confirmed that FGF-19, PLA2G10, and CPA2 had an AUC of about 0.7 or more in the discrimination between NC and sMCI, the discrimination between NC and MCI, and the discrimination between NC and AD, and that these proteins could discriminate between NC and sMCI and between NC and MCI well, and could also discriminate between NC and AD well. In addition, from the results in Tables 5 to 7, it was confirmed that the combinations of two or more of FGF-19, PLA2G10, CPA2, TIMP4, IGFBP1 and NEFL and TMPRSS15 showed an increase in AUC in the discrimination between NC and sMCI, the discrimination between NC and MCI, and the discrimination between NC and AD, as compared with when these proteins were used singly. Therefore, it was confirmed that the combinations of these proteins could discriminate between NC and sMCI and between NC and MCI well, and can also discriminate between NC and AD well, with higher discriminative ability than when they were used singly.

### Example 3: Screening for Biomarker (Lipid) of Mild Cognitive Impairment and Mild Alzheimer's Disease

### (1) Preparation of Specimen

The specimen group A and the specimen group B as described in Example 1 (1) were used as specimens.

### (2) Lipid Analysis

The mass spectrometer used was LCMS8060 system (Shimadzu Corporation), and measurements were made by Selected Reaction Monitoring (SRM) in the electrospray ion mode. A reversed-phase column such as Acquity UPLC BEH C8 column (1.7 μm, 2.1×100 mm, Waters) was used as the HPLC column. The mobile phase used was a two-liquid or three-liquid gradient in which ammonium carbonate, ammonium formate solution, acetonitrile, and isopropanol were appropriately mixed. In mass spectrometry, proton adducts or ammonium adducts were monitored, and ions that matched the target lipids were selected.

As the lipids, measurement was made for phosphatidylcholine (PC), phosphatidylethanolamine (PE), their lyso forms (lysophosphatidylcholine, LPC, and lysophosphatidylethanolamine, LPE), free fatty acids, triglycerides, and bile acids.

### (3) Analysis

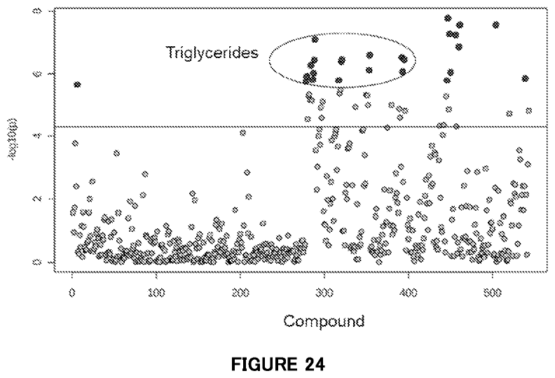

The data obtained by the software LabSolutions (Shimadzu Corporation) was saved in CSV format as the peak intensity or relative intensity of the lipids by the tool Traces developed in the laboratory, and FIGS. 23 and 24 were created using the ANOVA tool of Statistical Analysis (https://www.metaboanalyst.ca/MetaboAnalyst/upload/StatUploadView.xhtml) using MetaboAnalyst (https://www.metaboanalyst.ca/). FIGS. 25-1 to 25-2 were obtained by clicking on a target spot for ANOVA in FIG. 23 using MetaboAnalyst (https://www.metaboanalyst.ca/), and FIGS. 25-3 to 25-4 were obtained by clicking on a target spot for ANOVA in FIG. 24 using MetaboAnalyst (https://www.metaboanalyst.cal).

### (4) Results

The results were as shown in Table 8 and FIGS. 23 to 25.

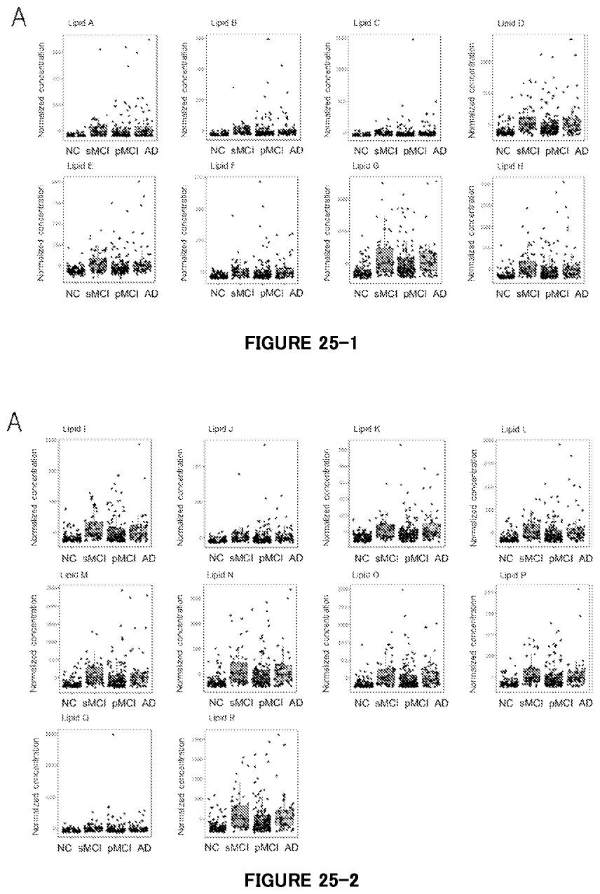

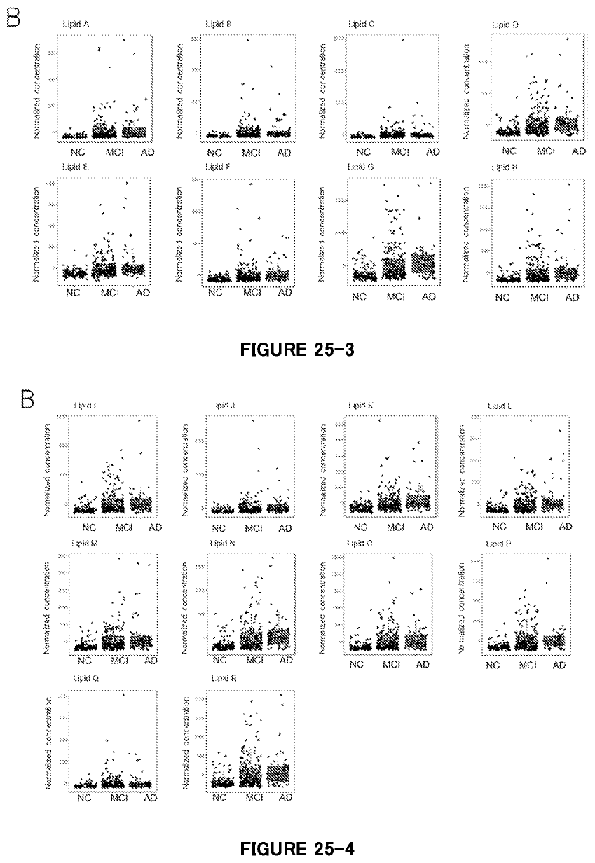

FIG. 23 shows the relative quantification values of the lipids (triglycerides) indicated in Table 8 in the plasma for the sMCI group, the pMCI group and the AD group to the quantification values thereof for the NC group; and FIG. 24 shows the relative quantification values of the lipids indicated in Table 8 in the plasma for the MCI group and the AD group to the quantification values thereof for the NC group. In this specification, each lipid indicated in Table 8 is sometimes indicated using the corresponding symbol in Table 8. In addition, the “triglyceride (name according to measurement method)” indicates a name in which the peak detected in mass spectrometry is presented according to the measurement method. Taking the indication “TAG(X:Y)_A_B” as an example, TAG denotes “triglyceride”; X denotes “total number of carbon atoms of constituent fatty acid”; Y denotes “number of unsaturated bonds”; A means that “in the first stage (Q1) of the mass spectrometry method, only ions with m/z of A were transmitted”; and B means that “in the next stage (Q3) of the mass spectrometry method, only ions with m/z of B were transmitted.” From FIG. 23, the sMCI and AD groups were confirmed to have significant differences in relative quantification values of the triglycerides indicated in Table 8, as compared with the NC group (p<0.00005, One-way ANOVA test). From FIG. 24, the MCI and AD groups were confirmed to have significant differences in relative quantification values of the triglycerides indicated in Table 8, as compared with the NC group (p<0.00005, One-way ANOVA test). The results of FIG. 25 showed that it is possible to detect NC and MCI (sMCI and pMCI) separately from each other and to separate and to detect NC and AD separately from each other by using the expression level of each of the 18 proteins indicated in Table 8 as the index. In addition, it was confirmed that, as compared with the NC group, the MCI groups (sMCI group and pMCI group) and the AD group were significantly high in expression levels of the lipids A to R.

TABLE 8
Biomarker (lipid) list

| | Triglyceride (Name | Triglyc- | |
|---|---|---|---|
| Sym- | according to | eride | Explanation of |
| bol | measurement method) | (Name) | triglyceride |
| Lipid A | TAG(58:1)_962.8_605.6 | TG | Component containing |
| | | 22:0_36:1 | fatty acid 22:0 (e.g., |
| | | | arachidic acid) among |
| | | | TG 58:1 |
| Lipid B | TAG(58:1)_962.8_661.5 | TG | Component containing |
| | | 18:0_40:1 | fatty acid 18:0 (e.g., |
| | | | stearic acid) among |
| | | | TG 58:1 |
| Lipid C | TAG(56:1)_934.8_633.5 | TG | Component containing |
| | | 18:0_38:1 | fatty acid 18:0 (e.g., |
| | | | stearic acid) among |
| | | | TG 56:1 |
| Lipid D | TAG(52:0)_880.8_607.5 | TG | Component containing |
| | | 16:0_36:0 | fatty acid 16:0 (e.g., |
| | | | palmitic acid) among |
| | | | TG 52:0 |
| Lipid E | TAG(54:0)_908.8_551.5 | TG | Component containing |
| | | 22:0_32:0 | fatty acid 22:0 (e.g., |
| | | | arachidic acid) among |
| | | | TG 54:0 |
| Lipid F | TAG(58:2)_960.8_661.6 | TG | Component containing |
| | | 18:1_40:1 | fatty acid 18:1 (e.g., |
| | | | oleic acid) among |
| | | | TG 58:2 |
| Lipid G | TAG(48:0)_824.8_551.5 | TG | Component containing |
| | | 16:0_32:0 | fatty acid 16:0 (e.g., |
| | | | palmitic acid) among |
| | | | TG 48:0 |
| Lipid H | TAG(52:0)_880.8_579.5 | TG | Component containing |
| | | 18:0_34:0 | fatty acid 18:0 (e.g., |
| | | | stearic acid) among |
| | | | TG 52:0 |
| Lipid I | TAG(48:0)_824.8_523.5 | TG | Component containing |
| | | 18:0_30:0 | fatty acid 18:0 (e.g., |
| | | | stearic acid) among |
| | | | TG 48:0 |
| Lipid J | TAG(56:1)_934.8_661.6 | TG | Component containing |
| | | 16:0_40:1 | fatty acid 16:0 (e.g., |
| | | | palmitic acid) among |
| | | | TG 56:1 |
| Lipid K | TAG(58:1)_962.8_663.6 | TG | Component containing |
| | | 18:1_40:0 | fatty acid 18:1 (e.g., |
| | | | oleic acid) among |
| | | | TG 58:1 |
| Lipid L | TAG(54:1)_906.8_607.5 | TG | Component containing |
| | | 18:1_36:0 | fatty acid 18:1 (e.g., |
| | | | oleic acid) among |
| | | | TG 54:1 |
| Lipid M | TAG(54:1)_906.8_605.5 | TG | Component containing |
| | | 18:0_36:1 | fatty acid 18:0 (e.g., |
| | | | stearic acid) among |
| | | | TG 54:1 |
| Lipid N | TAG(50:0)_852.8_551.5 | TG | Component containing |
| | | 18:0_32:0 | fatty acid 18:0 (e.g., |
| | | | stearic acid) among |
| | | | TG 50:0 |
| Lipid O | TAG(46:1)_794.8_521.5 | TG | Component containing |
| | | 16:0_30:1 | fatty acid 16:0 (e.g., |
| | | | palmitic acid) among |
| | | | TG 46:1 |
| Lipid P | TAG(48:0)_824.8_579.5 | TG | Component containing |
| | | 14:0_34:0 | fatty acid 14:0 (e.g., |
| | | | myristic acid) among |
| | | | TG 48:0 |
| Lipid Q | TAG(54:1)_906.8_633.6 | TG | Component containing |
| | | 16:0_38:1 | fatty acid 16:0 (e.g., |
| | | | palmitic acid) among |
| | | | TG 54:1 |
| Lipid R | TAG(50:0)_852.8_579.5 | TG | Component containing |
| | | 16:0_34:0 | fatty acid 16:0 (e.g., |
| | | | palmitic acid) among |
| | | | TG 50:0 |

### Example 4: Discrimination by Mild Cognitive Impairment and Mild Alzheimer's Disease Marker (Lipid)

### (1) ROC Analysis

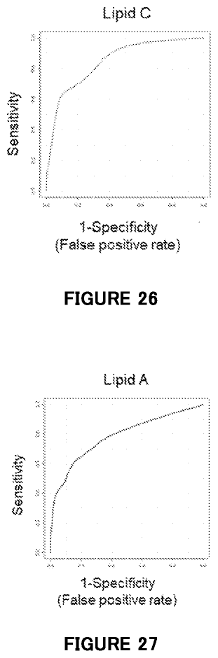

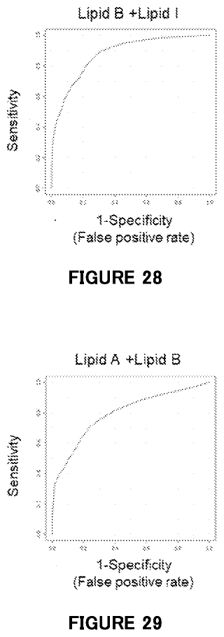

For the 18 lipids indicated in Table 8, for which significant differences were confirmed in Example 3, discrimination between NC and sMCI and discrimination between NC and MCI were analyzed using ROC curves (Tables 9 to 10 and FIGS. 26 to 27). Also, for combinations of the 18 lipids, discrimination between NC and sMCI and discrimination between NC and MCI were analyzed using ROC curves (Tables 11 to 12 and FIGS. 28 to 29). These analyses were performed using the statistical software described in Example 2 (1).

### (2) Results

The results were as shown in Tables 9 to 12 and FIGS. 26 to 29.

TABLE 9
Example of discrimination between NC
and sMCI by single biomarker (lipid)

| Symbol | AUC | 95% CI |
|---|---|---|
| Lipid A | 0.869 | 0.8-0.922 |
| Lipid B | 0.883 | 0.817-0.938 |
| Lipid C | 0.863 | 0.803-0.923 |
| Lipid D | 0.818 | 0.734-0.88 |
| Lipid E | 0.788 | 0.673-0.882 |
| Lipid F | 0.799 | 0.721-0.888 |
| Lipid G | 0.784 | 0.69-0.865 |
| Lipid H | 0.822 | 0.74-0.885 |
| Lipid I | 0.82 | 0.747-0.895 |
| Lipid J | 0.813 | 0.74-0.891 |
| Lipid K | 0.782 | 0.689-0.86 |
| Lipid L | 0.814 | 0.739-0.88 |
| Lipid M | 0.81 | 0.735-0.885 |
| Lipid N | 0.802 | 0.723-0.863 |
| Lipid O | 0.795 | 0.708-0.884 |
| Lipid P | 0.815 | 0.725-0.901 |
| Lipid Q | 0.796 | 0.718-0.875 |
| Lipid R | 0.793 | 0.708-0.86 |

TABLE 10
Example of discrimination between NC
and MCI by single biomarker (lipid)

| Symbol | AUC | 95% CI |
|---|---|---|
| Lipid A | 0.791 | 0.737-0.843 |
| Lipid B | 0.871 | 0.737-0.84 |
| Lipid C | 0.751 | 0.706-0.805 |
| Lipid D | 0.744 | 0.674-0.804 |
| Lipid E | 0.737 | 0.678-0.804 |
| Lipid F | 0.734 | 0.692-0.796 |
| Lipid G | 0.729 | 0.66-0.801 |
| Lipid H | 0.727 | 0.669-0.791 |
| Lipid I | 0.727 | 0.657-0.792 |
| Lipid J | 0.726 | 0.676-0.782 |
| Lipid K | 0.723 | 0.647-0.784 |
| Lipid L | 0.720 | 0.659-0.781 |
| Lipid M | 0.719 | 0.65-0.78 |
| Lipid N | 0.712 | 0.641-0.78 |
| Lipid O | 0.712 | 0.648-0.785 |
| Lipid P | 0.704 | 0.64-0.763 |
| Lipid Q | 0.708 | 0.654-0.772 |
| Lipid R | 0.711 | 0.637-0.774 |

TABLE 11
Example of discrimination between NC and
sMCI by combination of biomarkers (lipid)

| Combination | | | |
|---|---|---|---|
| No. | Combination | AUC | 95% CI |
| 101 | Lipid A and Lipid B | 0.887 | 0.822-0.946 |
| 102 | Lipid B and Lipid I | 0.888 | 0.824-0.936 |

TABLE 12
Example of discrimination between NC and
MCI by combination of biomarkers (lipid)

| Combination | | | |
|---|---|---|---|
| No. | Combination | AUC | 95% CI |
| 103 | Lipid A and Lipid B | 0.801 | 0.756-0.848 |

From the results in Tables 9 to 10, it was confirmed that the 18 lipids had an AUC of about 0.7 or more in the discrimination between NC and sMCI and the discrimination between NC and MCI, and that these lipids could discriminate singly between NC and sMCI well, and could also discriminate singly between NC and MCI well. In addition, from the results in Tables 11 to 12, it was confirmed that the combinations of two or more of the 18 lipids showed an increase in AUC in the discrimination between NC and sMCI and the discrimination between NC and MCI. Therefore, it was confirmed that the combinations of these lipids could discriminate between NC and sMCI and between NC and MCI well, with higher discriminative ability than when they were used singly.

### Example 5: Discrimination by Mild Cognitive Impairment and Mild Alzheimer's Disease Marker (Combination of Protein and Lipid)

### (1) ROC Analysis

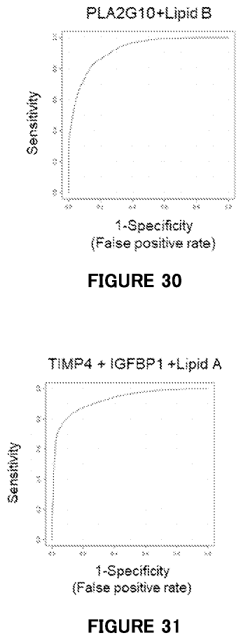

For combinations of the seven proteins for which significant differences were confirmed in Example 1 and the 18 lipids for which significant differences were confirmed in Example 3, discrimination between NC and sMCI and discrimination between NC and MCI were analyzed using ROC curves (Tables 13 to 14 and FIGS. 30 to 31). These analyses were performed in the same manner as in Examples 2 (1) and 3 (3).

### (2) Results

The results were as shown in Tables 13 to 14 and FIGS. 30 to 31.

TABLE 13
Example of discrimination between NC and sMCI by
combination of biomarkers (protein and lipid)

| Combination | | | | |
|---|---|---|---|---|
| No. | Protein | Lipid | AUC | 95% CI |
| 104 | PLA2G10 | Lipid B | 0.924 | 0.88-0.961 |
| 105 | PLA2G10 | Lipid C | 0.92 | 0.881-0.956 |
| 106 | PLA2G10 | Lipid H | 0.895 | 0.835-0.952 |
| 107 | PLA2G10 | Lipid I | 0.893 | 0.826-0.948 |

TABLE 14
Example of discrimination between NC and MCI by
combination of biomarkers (protein and lipid)

| Combination | | | | |
|---|---|---|---|---|
| No. | Protein | Lipid | AUC | 95% CI |
| 108 | FGF-19 | Lipid A | 0.831 | 0.764-0.878 |
| 109 | FGF-19 | Lipid B | 0.827 | 0.764-0.873 |
| 110 | PLA2G10 | Lipid A | 0.866 | 0.807-0.903 |
| 111 | PLA2G10 | Lipid B | 0.863 | 0.806-0.906 |
| 112 | PLA2G10 | Lipid C | 0.857 | 0.8-0.905 |
| 113 | PLA2G10 | Lipid D | 0.858 | 0.8-0.906 |
| 114 | TIMP4 | Lipid A | 0.909 | 0.87-0.942 |
| 115 | TIMP4 | Lipid B | 0.907 | 0.872-0.937 |
| 116 | TIMP4 | Lipid C | 0.901 | 0.858-0.939 |
| 117 | TIMP4 | Lipid D | 0.888 | 0.837-0.923 |
| 118 | NEFL | Lipid A | 0.868 | 0.821-0.904 |
| 119 | NEFL | Lipid B | 0.862 | 0.82-0.899 |
| 120 | FGF-19, | Lipid A | 0.909 | 0.868-0.943 |
| | TIMP4 | | | |
| 121 | PLA2G10, | Lipid A | 0.926 | 0.891-0.955 |
| | TIMP4 | | | |
| 122 | PLA2G10, | Lipid A | 0.906 | 0.868-0.936 |
| | IGFBP1 | | | |
| 123 | PLA2G10, | Lipid B | 0.906 | 0.874-0.935 |
| | IGFBP1 | | | |
| 124 | CPA2, TIMP4 | Lipid A | 0.913 | 0.879-0.942 |
| 125 | TIMP4, | Lipid A | 0.927 | 0.892-0.959 |
| | IGFBP1 | | | |
| 126 | TIMP4, | Lipid A | 0.921 | 0.887-0.949 |
| | NEFL | | | |
| 127 | IGFBP1, | Lipid A | 0.907 | 0.874-0.937 |
| | NEFL | | | |
| 128 | IGFBP1, | Lipid B | 0.907 | 0.872-0.942 |
| | NEFL | | | |
| 129 | FGF-19 | Lipid A, | 0.833 | 0.77-0.878 |
| | | Lipid B | | |
| 130 | PLA2G10 | Lipid A, | 0.869 | 0.814-0.909 |
| | | Lipid B | | |
| 131 | PLA2G10 | Lipid A, | 0.871 | 0.825-0.912 |
| | | Lipid I | | |
| 132 | TIMP4 | Lipid A, | 0.912 | 0.88-0.943 |
| | | Lipid B | | |
| 133 | TIMP4 | Lipid A, | 0.913 | 0.881-0.943 |
| | | Lipid C | | |
| 134 | TIMP4 | Lipid A, | 0.911 | 0.87-0.942 |
| | | Lipid D | | |
| 135 | TIMP4 | Lipid A, | 0.914 | 0.877-0.943 |
| | | Lipid I | | |
| 136 | TIMP4 | Lipid B, | 0.908 | 0.876-0.938 |
| | | Lipid C | | |
| 137 | TIMP4 | Lipid B, | 0.904 | 0.86-0.939 |
| | | Lipid D | | |
| 138 | NEFL | Lipid A, | 0.87 | 0.828-0.905 |
| | | Lipid B | | |

From the results in Tables 13 to 14, it was confirmed that the combinations of the seven proteins (FGF-19, PLA2G10, CPA2, TIMP4, IGFBP1, NEFL, and TMPRSS15) and the lipids indicated in Table 8 showed an increase in AUC in the discrimination between NC and sMCI and the discrimination between NC and MCI. Therefore, it was confirmed that the combinations of these proteins and lipids could discriminate between NC and sMCI and between NC and MCI well, with higher discriminative ability than when they were used singly.

1. A method for detecting mild cognitive impairment and/or mild Alzheimer's disease, comprising:
measuring the amount or concentration of a biomolecule in a biological sample from a test subject, wherein the biomolecule is a protein (biomolecule (a1)), and wherein the biomolecule (a1) is FGF-19.

2. The detection method according to claim 1, further comprising:
measuring the amount or concentration of a biomolecule other than the biomolecule (a1) in the biological sample from the test subject.

3. The detection method according to claim 2, wherein the biomolecule other than the biomolecule (a1) is a protein and/or a lipid.

4. The detection method according to claim 3, wherein the protein is proteins (biomolecule (a2)) selected from the group consisting of TIMP4, IGFBP1, NEFL and TMPRSS15.

5. The detection method according to claim 3, wherein the lipid is a triglyceride (biomolecule (b1)) free of fatty acid containing two or more unsaturated bonds.

6.-9. (canceled)

10. The detection method according to claim 1, further comprising:
determining the likelihood of suffering from mild cognitive impairment and/or mild Alzheimer's disease using the amount or concentration of the biomolecule in the biological sample from the test subject as an index.

11. The detection method according to claim 1, wherein the biological sample is a blood sample.

12.-22. (canceled)

23. A method for determining the progress state of the pathological condition of mild cognitive impairment and/or mild Alzheimer's disease, comprising:
measuring the amount or concentration of a biomolecule in a biological sample from a test subject, wherein the biomolecule is a protein (biomolecule (a1)), and wherein the biomolecule (a1) is FGF-19.

24. The determination method according to claim 23, further comprising:
measuring the amount or concentration of a biomolecule other than the biomolecule (a1) in the biological sample from the test subject.

25. The determination method according to claim 24, wherein the biomolecule other than the biomolecule (a1) is a protein and/or a lipid.

26.-28. (canceled)

29. The determination method according to claim 23, further comprising:
determining the degree of the progress state of the pathological condition of mild cognitive impairment and/or mild Alzheimer's disease using the amount or concentration of the biomolecule in the biological sample from the test subject as an index.

30. The determination method according to claim 23, wherein the biological sample is a blood sample.

31.-39. (canceled)

40. A kit for detection or diagnosis of mild cognitive impairment and/or mild Alzheimer's disease, comprising a means of quantifying proteins (biomolecule (a1)) selected from the group consisting of FGF-19, PLA2G10 and CPA2 and/or a triglyceride (biomolecule (b1)) free of fatty acid containing two or more unsaturated bonds, in a biological sample from a test subject.

41. The kit according to claim 40, further comprising a means of quantifying a biomolecule other than the biomolecule (a1) and the biomolecule (b1).

42. The kit according to claim 41, wherein the biomolecule other than the biomolecule (a1) and the biomolecule (b1) is a protein and/or a lipid, and preferably wherein the protein is proteins (biomolecule (a2)) selected from the group consisting of TIMP4, IGFBP1, NEFL and TMPRSS15.

43. (canceled)

44. The detection method according to claim 1, wherein the biomolecule (a1)) includes PLA2G10 and/or CPA2.

45. The detection method according to claim 5, wherein the triglyceride (biomolecule (b1)) free of fatty acid containing two or more unsaturated bonds lipid is one or more lipids selected from the group consisting of triglycerides listed in Table 1.

46. The determination method according to claim 23, wherein the biomolecule (a1)) includes PLA2G10 and/or CPA2.

47. The determination method according to claim 25, wherein the protein is proteins (biomolecule (a2)) selected from the group consisting of TIMP4, IGFBP1, NEFL and TMPRSS15.

48. The determination method according to claim 25, wherein the lipid is a triglyceride (biomolecule (b1)) free of fatty acid containing two or more unsaturated bonds, and more preferably wherein the lipid is one or more lipids selected from the group consisting of triglycerides listed in Table 1.
